(19) United States
(12) Patent Application Publication
(10) Pub. No.: US 2003/0199038 A1
(43) Pub. Date: Oct. 23, 2003

(54) Method for preparing polypeptide variants

(75) Inventors: Howard Brody, Davis, CA (US); Suzanne M. Otani, Elk Grove, CA (US); Joel R. Cherry, Davis, CA (US)

Correspondence Address: NOVOZYMES BIOTECH, INC., 1445 DREW AVE, DAVIS, CA, 95616, US

(73) Assignee: Novozymes Biotech, Inc., Davis, CA

(21) Appl. No.: 10/422,013

(22) Filed: Apr. 22, 2003

Related U.S. Application Data
(60) Provisional application No. 60/374,688, filed on Apr. 22, 2002.

(51) Int. Cl. C12P 21/02; C12N 9/02; C12N 9/10; C12N 9/16; C12N 9/88; C12N 9/90; C12N 1/16; C12N 15/74

(52) U.S. Cl. 435/69.1; 435/254.3; 435/189; 435/193; 435/196; 435/232; 435/233; 435/484

(57) ABSTRACT

The present invention relates to methods for preparing variants of a nucleotide sequence, comprising: (a) introducing into a population of filamentous fungal host cells: (i) one or more circular plasmids comprising a DNA sequence and a plasmid replicator mediating autonomous replication, wherein the one or more circularized plasmids are linearized by digestion of the DNA sequence and removal of a portion of the DNA sequence; and (ii) a library of DNA fragments comprising one or more mutations of the DNA sequence, wherein the fragments comprise at least two regions, one or more regions which are homologous to the 5′ region or the 3′ region of the gap in the linearized DNA sequence and/or plasmid sequence and one or more second regions which are homologous to the 5′ region or the 3′ region of the DNA fragments of the library; wherein the linearized plasmids and the DNA fragments recombine by in vivo recombination to produce a plurality of autonomously replicating plasmids comprising one or more variants of the DNA sequence; (b) cultivating the population of recombinant filamentous fungal cells in a medium suitable for growth; and (c) screening the population of recombinant filamentous fungal cells for variants of the DNA sequence contained on one or more autonomously replicating circularized plasmids.

### CROSS-REFERENCE TO RELATED APPLICATION

This application claims the benefit of U.S. Provisional Application No. 60/374,688, filed Apr. 22, 2002, which application is incorporated herein by reference.

### BACKGROUND OF THE INVENTION

1. Field of the Invention

The present invention relates to a method for preparing variants of a nucleic acid sequence by in vivo recombination.

2. Description of the Related Art

The advantages of producing biologically active polypeptides by cloning naturally occurring DNA sequences from microorganisms and expressing the DNA sequences in suitable host cells using recombinant DNA technology are well known in the art.

Novel polypeptide variants and mutants, particularly enzymes with improved properties such as specific activity, substrate specificity, pH-optimum, and temperature stability have been obtained by site-directed mutagenesis (see U.S. Pat. No. 4,518,584) and random mutagenesis (see, U.S. Pat. No. 4,894,331 and WO 93/01285). Site-directed mutagenesis results in substitution, deletion or insertion of specific amino acid residues, which have been chosen either on the basis of their type or on the basis of their location in the secondary or tertiary structure of the mature enzyme.

Since site-directed mutagenesis and random mutagenesis are cumbersome and time consuming methodologies, several alternative methods for the rapid preparation of modified polypeptides have been developed.

Weber et al., 1983, Nucleic Acids Research 11: 5661-5661, disclose a method for modifying genes by in vivo recombination between two homologous genes. A linear DNA sequence comprising a plasmid flanked to a DNA sequence encoding alpha-1 human interferon in the 5′-end and a DNA sequence encoding alpha-2 human interferon in the 3′-end is constructed and transfected into a recA positive strain of E. coli. Recombinants were identified and isolated using a resistance marker.

Pompon el al., 1989, Gene 83: 15-24, describe a method for shuffling gene domains of mammalian cytochrome P-450 by in vivo recombination of partially homologous sequences in Saccharomyces cerevisiae by transforming Saccharomyces cerevisiae with a linearized plasmid with filled-in ends, and a DNA fragment being partially homologous to the ends of said plasmid.

Stemmer, 1994, Proc. Natl. Acad. Sci. USA, 91: 10747-10751, and Stemmer, 1994, Nature 370: 389-391, disclose methods for shuffling homologous DNA sequences by an in vitro PCR method. One cycle of shuffling consists of digesting a pool of homologous genes with DNase I. The resulting small fragments are reassembled into full-length genes. Positive recombinant genes containing shuffled DNA sequences are selected from a DNA library based on their improved function. Positive recombinants can be used as the starting material for (an) other shuffling round(s).

U.S. Pat. No. 5,093,257 describes a method for producing hybrid polypeptides by in vivo recombination. Hybrid DNA sequences are produced by forming a circular plasmid comprising a replication sequence, a first DNA sequence encoding the amino-terminal portion of the hybrid polypeptide, a second DNA sequence encoding the carboxy-terminal portion of said hybrid polypeptide. The circular plasmid is transformed into a rec-positive microorganism in which the circular plasmid is amplified. This results in recombination of the circular plasmid mediated by the naturally occurring recombination mechanism of the rec-positive microorganism, which include prokaryotes such as Bacillus and E. coli, and eukaryotes such as Saccharomyces cerevisiae.

WO 00/24883 discloses methods of constructing and screening a library of polynucleotide sequences of interest in filamentous fungi by use of an episomal replicating AMA1-based plasmid vector.

Despite the availability of the above methods, there remains a need in the art for in vivo recombination methods for preparing variants of a DNA sequence in filamentous fungi.

The object of the present invention is to provide an improved method for preparing variants of a DNA sequence by in vivo recombination in filamentous fungi.

### SUMMARY OF THE INVENTION

The present invention relates to methods for preparing variants of a nucleotide sequence in a filamentous fungal host, comprising:

(a) introducing into a population of filamentous fungal host cells:

(i) one or more circular plasmids comprising a DNA sequence and a plasmid replicator mediating autonomous replication, wherein the one or more circularized plasmids are linearized by digestion of the DNA sequence and removal of a portion of the DNA sequence; and

(ii) a library of DNA fragments comprising one-or more mutations of the DNA sequence, wherein the fragments comprise at least two regions, one or more regions which are homologous to the 5′ region or the 3′ region of the gap in the linearized DNA sequence and/or plasmid sequence and one or more second regions which are homologous to the 5′ region or the 3′ region of the DNA fragments of the library;

wherein the linearized plasmids and the DNA fragments recombine by in vivo recombination to produce a plurality of autonomously replicating plasmids comprising one or more variants of the DNA sequence;

(b) cultivating the population of recombinant filamentous fungal cells in a medium suitable for growth; and

(c) screening the population of recombinant filamentous fungal cells for variants of the DNA sequence contained on one or more autonomously replicating circularized plasmids

### BRIEF DESCRIPTION OF THE FIGURES

FIGS. 1A and B show the genomic DNA sequence and the deduced amino acid sequence of an Aspergillus oryzae rdhA gene and encoded recombination protein (SEQ ID NOS:1 and 2, respectively).

FIGS. 2A, B, and C show the shows the genomic DNA sequence and the deduced amino acid sequence of an Aspergillus oryzae rdhB gene and encoded recombination protein (SEQ ID NOS:3 and 4, respectively).

FIGS. 3A, B, and C show the shows the genomic DNA sequence and the deduced amino acid sequence of an Aspergillus oryzae rdhD gene and encoded recombination protein (SEQ ID NOS:5 and 6, respectively).

FIG. 4 shows a restriction map of pPaHa3B.

FIG. 5 shows a restriction map of pSMO145.

FIG. 6 shows a restriction map of pToC202.

FIG. 7 shows a restriction map of pSMO146.

FIG. 8 shows a restriction map of pPH5.

FIG. 9 shows a restriction map of pPH7.

FIG. 10 shows a restriction map of pCWO13.

FIG. 11 shows the relative Humicola insolens cellobiohydralase activity of gap repaired transformants.

### DETAILED DESCRIPTION OF THE INVENTION

The present invention relates to methods for preparing variants of a nucleotide sequence in a filamentous fungal host, comprising: (a) introducing into a population of filamentous fungal host cells: (i) one or more circular plasmids comprising a DNA sequence and a plasmid replicator mediating autonomous replication, wherein the one or more circularized plasmids are linearized by digestion of the DNA sequence and removal of a portion of the DNA sequence, and (ii) a library of DNA fragments comprising one or more mutations of the DNA sequence, wherein the fragments comprise at least two regions, one or more regions which are homologous to the 5′ region or the 3′ region of the gap in the linearized DNA sequence and/or plasmid sequence and one or more second regions which are homologous to the 5′ region or the 3′ region of the DNA fragments of the library; wherein the linearized plasmids and the DNA fragments recombine by in vivo recombination to produce a plurality of autonomously replicating plasmids comprising one or more variants of the DNA sequence; (b) cultivating the population of recombinant filamentous fungal cells in a medium suitable for growth; and (c) screening the population of recombinant filamentous fungal cells for variants of the DNA sequence contained on one or more autonomously replicating circularized plasmids. This method, which we define here as gap repair, has a number of advantages over previously described methods.

An important advantage of the methods of the present invention is that they allow the shuffling of DNA fragments that are homologous with a DNA sequence of interest and the recovery of the resulting variants of the DNA sequence contained in autonomously replicating plasmids.

Another advantage of the methods of the present invention is that because of the efficient gap repair and high transformation frequency using the autonomous replicating plasmid, sufficient yields of gap repaired transformants can permit high throughput robotic screening similar to that performed in yeast.

A further advantage is that the present methods allow the construction of variant libraries in vivo in filamentous fungi. Previous methods for construction of variant libraries were dependent on the propagation of DNA in a host that allowed amplification of the said DNA, such as the propagation of plasmids containing bacterial replication sequences in E. coli, purification of the DNA, and transformation of the DNA into filamentous fungi. This method not only is much more labor intensive, but also is most typically accomplished by pooling of individual clones for plasmid purification. Such amplification, pooling, and transformation result in libraries in filamentous fungi that contain multiples of the original variants, increasing the screening required to ensure that all members of the original library are examined.

The present methods also allow the direct construction of autonomously replicating plasmids in vivo in filamentous fungi.

There is another important advantage of the methods of the present invention. In the yeast Saccharomyces cerevisiae the frequency of homologous recombination approaches 100%, allowing for very efficient gap repair as previously described by Pompon el al., 1989, Gene 83: 15-24. In contrast, the recombination frequency in many filamentous fungi, including Aspergillus oryzae and Aspergillus niger, usually varies between 0 and 5%, with most integration being random even when transformed with homologous DNA. For this reason, gap repair producing functional products is not expected in adequate numbers in filamentous fungi. Surprisingly, in the methods of the present invention, in vivo gap repair in Aspergillus oryzae indicate recombination resulting in functional products as a result of both perfect and imprecise homologous recombination within the overlap region shared between the gapped plasmid and linear DNA. The methods of the present invention may take advantage of this mode of recombination as over 90% functional recombination products can be obtained by having the recombination initiate within a non-functional region flanking the gap.

The term “shuffling” means recombination of nucleotide sequence(s) between two or more homologous DNA sequences resulting in recombines DNA sequences (i.e. DNA sequences having been subjected to a shuffling cycle) having a number of nucleotides exchanged, in comparison to the starting DNA sequences.

The term “recombination” is defined herein as the process wherein nucleic acids associate with each other in regions of homology, leading to interstrand DNA exchange between those sequences. For purposes of the present invention, homologous recombination is determined according to the procedures summarized by Paques and Haber, 1999, Microbiology and Molecular Biology Reviews 63: 349-404. The recombination may be homologous or non-homologous. “Homologous recombination” is defined herein as recombination in which no changes in the DNA sequences occurs within the regions of homology relative to the input DNA sequences. For perfect homologous recombination, the one or more regions should contain a sufficient number of nucleic acids, such as 100 to 1,500 base pairs, preferably 400 to 1,500 base pairs, and most preferably 800 to 1,500 base pairs, which are highly homologous with the corresponding nucleic acid sequence to enhance the probability of homologous recombination. “Non-homologous recombination” is defined herein as recombination where any mode of DNA repair incorporating strand exchange results in a DNA sequence different from any of the recombining sequences.

DNA Sequences

In the methods of the present invention, the DNA sequence may be any DNA sequence.

The DNA sequence preferably is selected from the group consisting of (a) a gene that encodes a polypeptide or an RNA; (b) a disrupted gene; (c) a partially deleted gene; (d) a regulatory control sequence; (e) a recombinantly manipulated version of a gene native or foreign to the filamentous fungal host cell; (f) a transposon; (g) a ribozyme; or (h) a portion of (a), (b), (c), (d), (e), (f) or (g). The DNA sequences may be wild-type DNA sequences, DNA sequences encoding variants or mutants, or modifications thereof, such as extended or elongated DNA sequences, and may also be the outcome of DNA sequences having been subjected to one or more cycles of shuffling (i.e. variant DNA sequences) according to the methods of the invention or any other method known in the prior art.

In a preferred embodiment, the DNA sequence comprises a gene encoding a polypeptide or an RNA. The polypeptide or RNA encoded by the DNA sequence may be native or heterologous to the fungal host cell of interest.

The term “polypeptide” is not meant herein to refer to a specific length of the encoded product and, therefore, encompasses peptides, oligopeptides, and proteins. The term “heterologous polypeptide” is defined herein as a polypeptide that is not native to the filamentous fungal cell; a native polypeptide in which modifications have been made to alter the native sequence; or a native polypeptide whose expression is quantitatively altered as a result of a manipulation of the filamentous fungal cell by recombinant DNA techniques. For example, a native polypeptide may be recombinantly produced by, for example, placing a gene encoding the polypeptide under the control of a strong promoter.

In the methods of the present invention, the DNA sequences may be either wild-type, variant or modified DNA sequences, such as a DNA sequences coding for wild-type, variant or modified enzymes, respectively.

The polypeptide may be an antibody, hormone, enzyme, receptor, reporter, selectable marker, or protein having biological activity. In a preferred embodiment, the polypeptide is an oxidoreductase, transferase, hydrolase, lyase, isomerase, or ligase. In a more preferred embodiment, the polypeptide is an aminopeptidase, amylase, carbohydrase, carboxypeptidase, catalase, cellulase, chitinase, cutinase, cyclodextrin glycosyltransferase, deoxyribonuclease, esterase, alpha-galactosidase, beta-galactosidase, glucoamylase, alpha-glucosidase, beta-glucosidase, invertase, laccase, lipase, mannosidase, mutanase, oxidase, pectinolytic enzyme, peroxidase, phospholipase, phytase, polyphenoloxidase, proteolytic enzyme, ribonuclease, transglutaminase, or xylanase. In another preferred embodiment, the polypeptide is secreted extracellularly.

The hormone or protein having biological activity may be insulin, ACTH, glucagon, somatostatin, somatotropin, thymosin, parathyroid hormone, pigmentary hormones, somatomedin, erythropoietin, luteinizing hormone, chorionic gonadotropin, hypothalamic releasing factors, antidiuretic hormones, thyroid stimulating hormone, relaxin, interferon, thrombopoietin (TPO), or prolactin.

The DNA sequence encoding a polypeptide of interest may be obtained from any prokaryotic, eukaryotic, or other source, if suitable for expression in a filamentous fungal cell. The techniques used to isolate or clone a DNA sequence of interest are known in the art and include isolation from genomic DNA, preparation from cDNA, or a combination thereof, as described above. The DNA sequence may be of genomic, cDNA, RNA, semisynthetic, synthetic origin, or any combinations thereof.

In the methods of the present invention, the polypeptide may also include a fused or hybrid polypeptide in which another polypeptide is fused at the N-terminus or the C-terminus of the polypeptide or fragment thereof. A fused polypeptide is produced by fusing a nucleic acid sequence (or a portion thereof) encoding one polypeptide to a nucleic acid sequence (or a portion thereof) encoding another polypeptide. Techniques for producing fusion polypeptides are known in the art, and include, ligating the coding sequences encoding the polypeptides so that they are in frame and expression of the fused polypeptide is under control of the same promoter(s) and terminator. The hybrid polypeptide may comprise a combination of partial or complete polypeptide sequences obtained from at least two different polypeptides wherein one or more may be heterologous to the mutant fungal cell.

In another preferred embodiment, the DNA sequence comprises a disrupted gene. The gene may be disrupted with any nucleic acid sequence. In a preferred embodiment, the gene is disrupted with a selectable marker gene. In a more preferred embodiment, the gene is disrupted with a selectable marker gene selected from the group consisting of amds (acetamidase), argB (ornithine carbamoyltransferase), bar (phosphinothricin acetyltransferase), hph (hygromycin phosphotransferase), niaD (nitrate reductase), pyrG (orotidine-5′-phosphate decarboxylase), sC (sulfate adenyltransferase), and trpC (anthranilate synthase), as well as equivalents thereof. Preferred for use in an Aspergillus cell are the amds and pyrG genes of Aspergillus nidulans or Aspergillus oryzae and the bargene of Streptomyces hygroscopicus. However, any selectable marker may be used if compatible with the filamentous fungal cell of choice.

In another preferred embodiment, the DNA sequence comprises a partially or fully deleted gene. Where the DNA sequence comprises a fully deleted gene, it will be understood that the nucleic acid sequence will contain regions upstream and downstream of the gene that are homologous with corresponding regions of the DNA fragments.

The DNA sequence comprising a disrupted or deleted gene may be constructed by using methods well known in the art, for example, insertions, disruptions, replacements, or deletions. The gene to be disrupted or deleted may be, for example, the coding region or a part thereof essential for activity, or the gene may contain a regulatory element required for expression of the coding region. An example of such a regulatory or control sequence may be a promoter sequence or a functional part thereof, i.e., a part which is sufficient for affecting expression of the nucleic acid sequence. Other control sequences for possible modification include, but are not limited to, a leader, polyadenylation sequence, propeptide sequence, signal sequence, transcription terminator, and transcriptional activator. See below for further discussion.

Disruption or deletion of the gene may be also accomplished by introduction, substitution, or removal of one or more nucleotides in the gene or a regulatory element required for the transcription or translation thereof. For example, nucleotides may be inserted or removed so as to result in the introduction of a stop codon, the removal of the start codon, or a change of the open reading frame.

An example of a convenient way to disrupt or delete a gene is based on techniques of gene replacement, gene deletion, or gene disruption. For example, in the gene disruption method, a nucleic acid sequence corresponding to the endogenous gene or gene fragment of interest is mutagenized in vitro to produce a defective nucleic acid sequence which is then transformed into the parent cell to produce a defective gene. By homologous recombination, the defective nucleic acid sequence replaces the endogenous gene or gene fragment. It may be desirable that the defective gene or gene fragment also encodes a marker, which may be used for selection of transformants in which the nucleic acid sequence has been modified or destroyed. The selectable marker gene may be used to achieve the disruption. The defective nucleic acid sequence may be a simple disruption of the endogenous sequence with a selectable marker gene. Alternatively, the defective nucleic acid sequence may contain an insertion or deletion of the endogenous sequence, or a portion thereof, in addition to the disruption with the selectable marker gene. Furthermore, the defective nucleic acid sequence may contain an insertion or deletion of the endogenous sequence, or a portion thereof, and the selectable marker gene is not involved in the modification but is used as a selectable marker for identifying transformants containing the defective gene.

In another preferred embodiment, the DNA sequence comprises a regulatory control sequence. The regulatory control sequence can be any control sequence, including, but not limited to, a promoter, signal sequence, leader, polyadenylation sequence, propeptide sequence, consensus translational initiator sequence, signal peptide sequence, and transcription terminator.

In another preferred embodiment, the DNA sequence comprises a recombinantly manipulated version of a gene native or foreign to the filamentous fungal cell.

In another preferred embodiment, the DNA sequence comprises a transposon. The term “transposon” is defined herein as mobile DNA sequence that can move from one site in a genome to another, or between different chromosomes (see Plant Pathology 534 (Gen CB 534) Fungal Genetics Spring 2001). There are two basic types of transposable elements in all organisms: (1) DNA sequences which move themselves to a new location as DNA (2) DNA sequences which move to a new location via an RNA intermediate. Transposons can: (1) inactivate genes (2) re-activate pseudogenes (genes which are unable to code for proteins) because they have promoter sequences 3) change expression of genes if they insert in regulatory regions. Transposons can promote rearrangements of the genome either directly or indirectly: (a) directly—transposition event may cause deletions or inversions (b) indirectly—transposons serve as substrates for recombination—“portable regions of homology”—provide sites for reciprocal recombination.

Examples of transposons include, but are not limited to, P elements, LINES, SINES, Ty1, gypsy, Fot1, hAT, Restless, Guest, elements, tn10, Tad-1, Afut-1, and the retrotransposons MAGGY Ty3 and Ty5.

Plasmid Replicator

In the methods of the present invention, the plasmid replicator may be any plasmid replicator mediating autonomous replication which functions in a filamentous fungal cell. The term “plasmid replicator” is defined herein as a sequence that enables a plasmid or vector to replicate independent of chromosomal replication. Replicators often consist of sequences that do not represent authentic genomic replicators. Their mode of function in most cases are not understood. Often these plasmids occur spontaneously and are not recognized by mitotic mechanisms and are quickly lost lacking selective pressure.

Examples of a plasmid replicator useful in a filamentous fungal cell is AMA1 and ANS1 (Gems et al., 1991, Gene 98:61-67; Cullen et al., 1987, Nucleic Acids Research 15: 9163-9175; WO 00/24883). Isolation of the AMA1 gene and construction of plasmids or vectors comprising the gene can be accomplished according to the methods disclosed in WO 00/24883.

Plasmids

The plasmid or plasmids may be any plasmid or vector that may conveniently be subjected to recombinant DNA procedures. The plasmid comprising the DNA sequence may be prepared by ligating the DNA sequence into a suitable plasmid, or by any other suitable method. The choice of plasmid will often depend on the filamentous fungal host cell into which it is to be introduced. In the methods of the present invention, the plasmid is an autonomously replicating plasmid, i.e. a plasmid which exists as an extrachromosomal entity, the replication of which is independent of chromosomal replication.

The linearizing of the plasmid(s) can be directed toward any site within the plasmid. The plamid(s) may be linearized by any suitable methods known in the art, for example, digestion with a restriction enzyme. The linearized ends of the plasmid may be filled-in with nucleotides as described in Pompon et al., 1989, supra. However, it is preferred not to fill in the linearized ends as it might create a frameshift.

To facilitate the screening process, the plasmid is preferably an expression vector in which the DNA sequence in question is operably linked to additional segments required for transcription of the DNA. In general, the expression vector is derived from a plasmid, a cosmid or a bacteriophage, or may contain elements of any or all of these. For purposes of the present invention, the terms “plasmid” and “vector” are used interchangeably.

The DNA sequence will generally be operably linked to one or more regulatory control sequences which direct the expression of the coding sequence in a suitable host cell under conditions compatible with the control sequences. The term “expression” will be understood to include any step involved in the production of the polypeptide including, but not limited to, transcription, post-transcriptional modification, translation, post-translational modification, and secretion. The term “operably linked” indicates that the segments are arranged so that they function in concert for their intended purposes, e.g., transcription initiates in a promoter and proceeds through the DNA sequence coding for the polypeptide in question.

The DNA sequence may be manipulated in a variety of ways to provide for expression of the polypeptide. Manipulation of the DNA sequence prior to its insertion into a plasmid or vector may be desirable or necessary depending on the DNA sequence, expression vector, and/or filamentous fungal host cell. The techniques for modifying nucleic acid sequences utilizing recombinant DNA methods are well known in the art.

The term “regulatory control sequences” is defined herein to include all components which are necessary or advantageous for the expression of a polypeptide of the present invention. Each control sequence may be native or foreign to the nucleic acid sequence encoding the polypeptide. Such control sequences include, but are not limited to, a leader, polyadenylation sequence, propeptide sequence, consensus translational initiator sequence of the present invention, signal peptide sequence, and transcription terminator. At a minimum, the control sequences include transcriptional and translational stop signals. The control sequences may be provided with linkers for the purpose of introducing specific restriction sites facilitating ligation of the control sequences with the coding region of the nucleic acid sequence encoding a polypeptide.

The control sequence may be an appropriate promoter sequence, a nucleic acid sequence which is recognized by a host cell for expression of the DNA sequence. The promoter sequence contains transcriptional control sequences which mediate the expression of the polypeptide. The promoter may be any nucleic acid sequence which shows transcriptional activity in the filamentous fungal host cell of choice including mutant, truncated, and hybrid promoters, and may be obtained from genes encoding extracellular or intracellular polypeptides either homologous or heterologous to the host cell.

Examples of suitable promoters for directing the transcription of the DNA sequence in a filamentous fungal host cell are promoters obtained from the genes for Aspergillus oryzae TAKA amylase, Rhizomucor miehei aspartic proteinase, Aspergillus niger neutral alpha-amylase, Aspergillus niger acid stable alpha-amylase, Aspergillus niger or Aspergillus awamori glucoamylase (glaA), Rhizomucor miehei lipase, Aspergillus oryzae alkaline protease, Aspergillus oryzae triose phosphate isomerase, Aspergillus nidulans acetamidase, Fusarium venenatum amyloglucosidase, Fusarium oxysporum trypsin-like protease (WO 96/00787), as well as the NA2-tpi promoter (a hybrid of the promoters from the genes for Aspergillus niger neutral alpha-amylase and Aspergillus oryzae triose phosphate isomerase); and mutant, truncated, and hybrid promoters thereof.

The control sequence may be a suitable transcription terminator sequence, a sequence recognized by a host cell to terminate transcription. The terminator sequence is operably linked to the 3′ terminus of the DNA sequence. Any terminator which is functional in the filamentous fungal host cell of choice may be used in the present invention.

Preferred terminators for filamentous fungal host cells are obtained from the genes for Aspergillus oryzae TAKA amylase, Aspergillus niger glucoamylase, Aspergillus nidulans anthranilate synthase, Aspergillus niger alpha-glucosidase, and Fusarium oxysporum trypsin-like protease.

The control sequence may also be a suitable leader sequence, a non-translated region of an mRNA which is important for translation by the filamentous fungal host cell. The leader sequence is operably linked to the 5′-terminus of the DNA sequence. Any leader sequence that is functional in the host cell of choice may be used in the present invention.

Preferred leaders for filamentous fungal host cells are obtained from the genes for Aspergillus oryzae TAKA amylase and Aspergillus nidulans triose phosphate isomerase.

The control sequence may also be a polyadenylation sequence, a sequence operably linked to the 3′ terminus of the DNA sequence and which, when transcribed, is recognized by the host cell as a signal to add polyadenosine residues to transcribed mRNA. Any polyadenylation sequence which is functional in the filamentous fungal host cell of choice may be used in the present invention.

Preferred polyadenylation sequences for filamentous fungal host cells are obtained from the genes for Aspergillus oryzae TAKA amylase, Aspergillus niger glucoamylase, Aspergillus nidulans anthranilate synthase, Fusarium oxysporum trypsin-like protease, and Aspergillus niger alpha-glucosidase.

The control sequence may also be a signal peptide coding region that codes for an amino acid sequence linked to the amino terminus of a polypeptide and directs the encoded polypeptide into the cell's secretory pathway. The 5′-end of the coding sequence of the DNA sequence encoding a polypeptide may inherently contain a signal peptide coding region naturally linked in translation reading frame with the segment of the coding region which encodes the secreted polypeptide. Alternatively, the 5′-end of the coding sequence may contain a signal peptide coding region which is foreign to the coding sequence. The foreign signal peptide coding region may be required where the coding sequence does not naturally contain a signal peptide coding region. Alternatively, the foreign signal peptide coding region may simply replace the natural signal peptide coding region in order to enhance secretion of the polypeptide. However, any signal peptide coding region which directs the expressed polypeptide into the secretory pathway of a filamentous fungal host cell of choice may be used in the present invention.

Effective signal peptide coding regions for filamentous fungal host cells are the signal peptide coding regions obtained from the genes for Aspergillus oryzae TAKA amylase, Aspergillus niger neutral amylase, Aspergillus niger glucoamylase, Rhizomucor miehei aspartic proteinase, Humicola insolens cellulase, and Humicola lanuginosa lipase.

The control sequence may also be a propeptide coding region that codes for an amino acid sequence positioned at the amino terminus of a polypeptide. The resultant polypeptide is known as a proenzyme or propolypeptide (or a zymogen in some cases). A propolypeptide is generally inactive and can be converted to a mature active polypeptide by catalytic or autocatalytic cleavage of the propeptide from the propolypeptide. The propeptide coding region may be obtained from the genes for Rhizomucormiehei aspartic proteinase and Myceliophthora thermophila laccase (WO 95/33836).

Where both signal peptide and propeptide regions are present at the amino terminus of a polypeptide, the propeptide region is positioned next to the amino terminus of a polypeptide and the signal peptide region is positioned next to the amino terminus of the propeptide region.

It may also be desirable to add regulatory sequences which allow the regulation of the expression of the DNA sequence relative to the growth of the host cell. Examples of regulatory systems are those which cause the expression of the gene to be turned on or off in response to a chemical or physical stimulus, including the presence of a regulatory compound. In filamentous fungi, the TAKA alpha-amylase promoter, Aspergillus niger glucoamylase promoter, and Aspergillus oryzae glucoamylase promoter may be used as regulatory sequences. Other examples of regulatory sequences are those which allow for gene amplification. In eukaryotic systems, these include the dihydrofolate reductase gene which is amplified in the presence of methotrexate, and the metallothionein genes which are amplified with heavy metals. In these cases, the nucleic acid sequence encoding the polypeptide would be operably linked with the regulatory sequence.

DNA Fragments

The library of DNA fragments to be randomly combined (or “shuffled”) with homologous regions in the linearized plasmid(s) by in vivo recombination may be prepared by any suitable method. For instance, the DNA fragment may be prepared by PCR amplification (polymerase chain reaction) of a plasmid or plasmid comprising the DNA sequence, using specific primers, for instance as described in U.S. Pat. No. 4,683,202 or Saiki et al., 1988, Science 239:487-491. The DNA fragment may also be isolated from a plasmid or plasmid comprising the desired DNA sequence by digestion with restriction enzymes, followed by isolation using, for example, electrophoresis.

The DNA fragment may alternatively be prepared synthetically by established standard methods, e.g. the phosphoamidite method described by Beaucage and Caruthers, 1981, Tetrahedron Letters 22: 1859-1869, or the method described by Matthes et al., (1984), EMBO Journal 3: 801-805. According to the phosphoamidite method, oligonucleotides are synthesized, for example, in an automatic DNA synthesizer, purified, annealed, ligated, and cloned into suitable plasmids.

Furthermore, the DNA fragment may be of mixed synthetic and genomic, mixed synthetic and cDNA or mixed genomic and cDNA origin prepared by ligating fragments of synthetic, genomic or cDNA origin (as appropriate), the fragments corresponding to various parts of the entire DNA sequence, in accordance with standard techniques.

The library of DNA fragments comprise one or more mutations of the DNA sequence, wherein the fragments comprise at least two regions, one or more regions which are homologous to the 5′ region or the 3′ region of the gap in the linearized DNA sequence and/or plasmid sequence and one or more second regions which are homologous to the 5′ region or the 3′ region of the DNA fragments of the library.

The regions of the DNA fragment may be any sequence that is homologous with the DNA sequence and/or plasmid sequence.

In a preferred embodiment, the two or more regions of the DNA fragment are a 5′ region and/or a 3′ region that flank (a) a gene that encodes a polypeptide or an RNA; (b) a gene disrupted with a third nucleic acid sequence; (c) a partially deleted gene; (d) a regulatory control sequence; (e) a recombinantly manipulated version of a gene native or foreign to the filamentous fungal host cell; (f) a transposon; (g) a ribozyme; or (h) a portion of (a), (b), (c), (d), (e), (f) or (g).

In another preferred embodiment, the two or more regions of the DNA fragment are a 5′ region and/or a 3′ region of (a) a gene that encodes a polypeptide or an RNA; (b) a gene disrupted with a third nucleic acid sequence; (c) a partially deleted gene; (d) a regulatory control sequence; (e) a recombinantly manipulated version of a gene native or foreign to the filamentous fungal host cell; (f) a transposon; (g) a ribozyme; or (h) a portion of (a), (b), (c), (d), (e), (f) or (g).

In another preferred embodiment, the one or more regions of the DNA fragment that are homologous to the DNA sequence are part of a gene native or foreign to the filamentous fungal host cell.

In a preferred embodiment of the present invention, the DNA fragment fragments are prepared under conditions resulting in a low, medium or high random mutagenesis frequency. To obtain low mutagenesis frequency the DNA sequence(s) (comprising the DNA fragment(s)) may be prepared by a standard PCR amplification method (U.S. Pat. No. 4,683,202 or Saiki et al., 1988, Science 239: 487-491). A medium or high mutagenesis frequency may be obtained by performing the PCR amplification under conditions which reduce the fidelity of replicaton by the thermostable polymerase and increase the misincorporation of nucleotides, for instance as described by Deshler, 1992, GATA 9: 103-106; Leung et al., 1989, BioTechniques 1: 11-15.

The PCR amplification (i.e. according to this embodiment also DNA fragment mutation) may be combined with a mutagenesis step using a suitable physical or chemical mutagenizing agent, e.g., one which induces transitions, transversions, inversions, scrambling, deletions, and/or insertions.

In a preferred embodiment, the DNA fragment(s) to be shuffled preferably have a length of from about 30 bp to 8 kb, more preferably about 40 bp to 6 kb, even more preferably about 80 bp to 4 kb, and most preferably about 100 bp to 2 kb, to be able to interact optimally with the linearized plasmid.

Filamentous Fungal Host Cells

The filamentous fungal host cell, into which the mixture of plasmid/fragment DNA sequences are to be introduced, may be any filamentous fungal cell useful in the methods of the present invention. A “recombination filamentous fungal cell” is defined herein as a cell capable of mediating shuffling of a number of homologous DNA sequences.

“Filamentous fungi” include all filamentous forms of the subdivision Eumycota and Oomycota (as defined by Hawksworth et al., 1995, supra). The filamentous fungi are characterized by a mycelial wall composed of chitin, cellulose, glucan, chitosan, mannan, and other complex polysaccharides. Vegetative growth is by hyphal elongation and carbon catabolism is obligately aerobic. In contrast, vegetative growth by yeasts such as Saccharomyces cerevisiae is by budding of a unicellular thallus and carbon catabolism may be fermentative.

In a preferred embodiment, the filamentous fungal host cell is an Acremonium, Aspergillus, Fusarium, Humicola, Mucor, Myceliophthora, Neurospora, Penicillium, Thielavia, Tolypocladium, or Trichoderma cell.

In a more preferred embodiment, the filamentous fungal host cell is an Aspergillus awamori, Aspergillus foetidus, Aspergillus japonicus, Aspergillus nidulans, Aspergillus niger or Aspergillus oryzae cell. In another most preferred embodiment, the filamentous fungal host cell is a Fusarium bactridioides, Fusarium cerealis, Fusarium crookwellense, Fusarium culmorum, Fusarium graminearum, Fusarium graminum, Fusarium heterosporum, Fusarium negundi, Fusarium oxysporum, Fusarium reticulatum, Fusarium roseum, Fusarium sambucinum, Fusarium sarcochroum, Fusarium sporotrichioides, Fusarium sulphureum, Fusarium torulosum, Fusarium trichothecioides, or Fusarium venenatum cell. In another most preferred embodiment, the filamentous fungal host cell is a Humicola insolens, Humicola lanuginosa, Mucor miehei, Myceliophthora thermophila, Neurospora crassa, Penicilliumpurpurogenum, Thielavia terrestris, Trichoderma harzianum, Trichoderma koningii, Trichoderma longibrachiatum, Trichoderma reesei, or Trichoderma viride cell.

In another most preferred embodiment, the Aspergillus cell is an Aspergillus oryzae cell.

In another most preferred embodiment, the Aspergillus cell is an Aspergillus niger cell.

In another most preferred embodiment, the Fusarium venenatum cell is Fusarium venenatum A3/5, which was originally deposited as Fusarium graminearum ATCC 20334 and recently reclassified as Fusarium venenatum by Yoder and Christianson, 1998, Fungal Genetics and Biology 23: 62-80 and O'Donnell et al., 1998, Fungal Genetics and Biology 23: 57-67; as well as taxonomic equivalents of Fusarium venenatum regardless of the species name by which they are currently known. In another preferred embodiment, the Fusarium venenatum cell is a morphological mutant of Fusarium venenatum A3/5 or Fusarium venenatum ATCC 20334, as disclosed in WO 97/26330.

Fungal cells may be transformed by a process involving protoplast formation, transformation of the protoplasts, and regeneration of the cell wall in a manner known per se. Suitable procedures for transformation of Aspergillus host cells are described in EP 238 023 and Yelton et al., 1984, Proceedings of the National Academy of Sciences USA 81: 1470-1474. Suitable methods for transforming Fusarium species are described by Malardier et al., 1989, Gene 78:147-156 and WO 96/00787.

In vivo Recombination

The methods of the present invention result in a high level of mixing of homologous genes or variants. A large number of variants or homologous genes can be mixed in one transformation. The mixing of improved variants or wild type genes followed by screening increases multi-fold the number of further improved variants compared to doing only random mutagenesis (for review see Kuchner, K. and Arnold, F. H. 1997. Directed evolution of enzyme catalysts. TIBTech 15:523-530). Random mutagenesis introduces mutations into a target DNA sequence, creating deleterious mutations much more frequently than beneficial ones. In iterative rounds of such mutagenesis, deleterious mutations accumulate more rapidly than beneficial ones, effectively masking the identification of beneficial mutations during screening. The random recombination between two or more homologous DNA sequences that contain multiple single nucleotide changes in their DNA sequences potentially allows all those nucleotide changes contained in one variant to be separated from one another and to be randomly mixed with any mutations present on other variants. This shuffling of mutations allows a means by which mutations from different parent sequences can be combined with each other randomly, The result of utilizing this method is an increased probability of combining nucleotide changes in a single DNA sequence.

Recombination of multiple overlapping fragments is possible with a high efficiency increasing the mixing of variants or homologous genes using the in vivo recombination method. An overlap as small as 30 bp is sufficient for recombination which may be utilized for very easy domain shuffling of even distantly related genes. In domain shuffling, larger blocks of non-homologous DNA are randomly assorted by means of stretches of homology at their termini.

In methods of the present invention, the term “positive polypeptide variants” means resulting polypeptide variants possessing a functional property or properties which have been improved in comparison to the polypeptides producible from the corresponding input DNA sequences. Examples, of such improved properties can be as different as e.g. biological activity, enzyme washing performance, antibiotic resistance etc. If the improved functional property of the polypeptide is not sufficiently good after one cycle of shuffling, the variant DNA sequence may be subjected to another cycle ad infinitum.

In a preferred embodiment, at least one shuffling cycle is a backcrossing cycle with the initially used DNA fragment or fragments, which may be the wild-type DNA fragment. This eliminates non-essential mutations. Non-essential mutations may also be eliminated by using wild-type DNA fragments as the initially used input DNA material.

However, the method of the present invention will in most cases lead to the replacement of a considerable number of amino acid and may in certain cases even alter the structure of one or more polypeptide domains (i.e. a folded unit of polypeptide structure).

According to the present invention more than two DNA sequences are shuffled at the same time. Actually any number of different DNA fragments and homologous polypeptides comprised in suitable plasmids may be shuffled at the same time. This is advantageous as a vast number of quite different variants can be made rapidly without an abundance of iterative procedures.

When recombining many fragments from the same region, multiple overlapping of the fragments will increase mixing by itself, but it is also important to have a relative high random mixing in overlapping regions in order to mix closely located variants/differences.

An overlap as small as 30 bp between two fragments is sufficient to obtain a very efficient recombination. Therefore, overlapping in the range from 30 to 5000 bp, preferably from 30 bp to 500 bp, especially 30 bp to 100 bp is suitable in the methods of the present invention.

In this embodiment of the present invention, preferably 2 or more overlapping fragments, more preferably 2 to 50 overlapping fragments, and most preferably 2 to 10 overlapping fragments may advantageously be used as DNA fragments in a shuffling cycle.

Besides increasing the mixing of genes, this is a very useful method for domain shuffling by creating small overlaps between DNA fragments from different domains and screen for the best combination. For example, in the case of three DNA fragments the overlapping regions may be as follows: the first end of the first fragment overlaps the first end of the linearized plasmid, the first end of the second fragment overlaps the second end of the first fragment, and the second end of the second fragment overlaps the first end of the third fragment, the first end of the third fragment overlaps (as stated above) the second end of the second fragment, and the second end of the third fragment overlaps the second end of the linearized plasmid.

It is understood that when using two or more DNA fragments as starting material, it is preferred to have continuous overlaps between the ends of the plasmid and the DNA fragments.

Even though it is preferred to shuffle homologous DNA sequences in the form of DNA fragment(s) and linearized plasmid(s), it is also possible to shuffle two or more linearized plasmids comprising homologous DNA sequences encoding polypeptides. However, in such a case it is important to linearize the plasmids at different sites.

In a further embodiment of the invention two or more linearized plasmids and one or more homologous DNA fragments are used as the starting material to be shuffled. The ratio between the linearized plasmid(s) and homologous DNA fragment(s) preferably lie in the range from 20:1 to 1:50, preferable from 2:1 to 1:10 (mol plasmid:mol fragments) with the specific concentrations being from 1 μM to 10 M of the DNA.

The linearized plasmids may be gapped in such a way that the overlap between the fragments is deleted in the plasmid. The repair of the gap in the plasmid then requires that the fragments recombine with one another in addition to recombining with the ends of the gapped plasmid in order to reconstitute a circular, autonomously replicating plasmid. In a preferred embodiment, the linearization of the plasmid or vector creates a sufficient gap in the coding sequence of the DNA sequence to force the homologous recombination of the DNA fragments with the corresponding regions of the DNA sequence. As mentioned earlier, gap repair producing functional products is not expected in adequate numbers in filamentous fungi. However, under the methods of the present invention, in vivo gap repair in Aspergillus oryzae indicates recombination resulting in functional products as a result of both perfect and imprecise homologous recombination within the overlap region shared between the gapped plasmid and linear DNA. Under this mode of recombination, over 90% functional recombination products can be obtained by having the recombination initiate within a non-functional, non-target region flanking the gap. Incorporation into a self-replicating plasmid increases the transformation frequency up to 4 orders of magnitude permitting organisms with inefficient rates of recombination, to achieve sufficient enough transformation for high throughput screening.

Methods of Cultivation

In the methods of the present invention, the recombinant filamentous fungal host cells are cultivated in a nutrient medium suitable for growth of the cell or the production of the polypeptide variants of interest using methods known in the art. For example, the filamentous fungal cell may be cultivated by shake flask cultivation, and small-scale or large-scale fermentation (including continuous, batch, fed-batch, or solid state fermentations) in laboratory or industrial fermentors performed in a suitable medium and under conditions allowing the polypeptide to be expressed and/or isolated. The cultivation takes place in a suitable nutrient medium comprising carbon and nitrogen sources and inorganic salts, using procedures known in the art. Suitable media are available from commercial suppliers or may be prepared according to published compositions (e.g., in catalogues of the American Type Culture Collection). If the polypeptide is secreted into the nutrient medium, the polypeptide can be recovered directly from the medium. If the polypeptide is not secreted, it can be recovered from cell lysates.

The polypeptide variants may be detected using methods well known in the art that are specific for the polypeptides. These detection methods may include use of specific antibodies, formation of an enzyme product, or disappearance of an enzyme substrate. For example, an enzyme assay may be used to determine the activity of the polypeptide as described herein.

The resulting polypeptide variants may be recovered by methods known in the art. For example, the polypeptide may be recovered from the nutrient medium by conventional procedures including, but not limited to, centrifugation, filtration, extraction, spray-drying, evaporation, or precipitation.

The polypeptide variants may be purified by a variety of procedures known in the art including, but not limited to, chromatography (e.g., ion exchange, affinity, hydrophobic, chromatofocusing, and size exclusion), electrophoretic procedures (e.g., preparative isoelectric focusing), differential solubility (e.g., ammonium sulfate precipitation), SDS-PAGE, or extraction (see, e.g., Protein Purification, J.-C. Janson and Lars Ryden, editors, VCH Publishers, New York, 1989).

Screening of Nucleic Acid Variants

The screening method to be used for identifying positive variants depend on the desired improved property of the polypeptide variant or variant of the DNA sequence in question. The improved property of interest can be, but is not limited to, thermostability, thermolability, protease-resistance, pH optimum, pH stability, altered substrate specificity, and increased promoter activity.

The resulting variant DNA sequences (i.e. shuffled DNA sequences), will have a number of nucleotide(s) exchanged, which results in replacement of at least one amino acid within the corresponding polypeptide variant, when compared with the parent polypeptide. It is to be understood that silent mutations are also contemplated (i.e. nucleotide exchange which does not result in changes in the amino acid sequence).

If, for instance, the polypeptide in question is an enzyme and the desired improved functional property is the wash performance, the screening may conveniently be performed by use of a filter assay based on the following principle: The recombination host cell is incubated on a suitable medium and under suitable conditions for the enzyme to be secreted, the medium being provided with a double filter comprising a first protein-binding filter and on top of that a second filter exhibiting a low protein binding capability. The recombination host cell is located on the second filter. Subsequent to the incubation, the first filter comprising the enzyme secreted from the recombination host cell is separated from the second filter comprising said cells. The first filter is subjected to screening for the desired enzymatic activity and the corresponding microbial colonies present on the second filter are identified.

The filter used for binding the enzymatic activity may be any protein binding filter e.g. nylon or nitrocellulose. The top filter carrying the colonies of the expression organism may be any filter that has no or low affinity for binding proteins e.g. cellulose acetate. The filter may be pre-treated with any of the conditions to be used for screening or may be treated during the detection of enzymatic activity.

The enzymatic activity may be detected by a dye, fluorescence, precipitation, pH indicator, IR-absorbance or any other known technique for detection of enzymatic activity.

The detecting compound may be immobilized by any immobilizing agent e.g. agarose, agar, gelatine, polyacrylamide, starch, filter paper, cloth; or any combination of immobilizing agents.

In the case of variants of a DNA sequence, the variant sequences can be subjected to PCR, isolated, and sequenced using conventional methods to ascertain the nature of the changes in the sequence. Alternately, a desired change in a DNA sequence may be screened for any cell phenotype that it alters, such as plasmid copy number, protein expression level, level of antibiotic resistance, cell wall properties such as resistance to organic solvents or detergents, increased RNA stability, catalytic nucleic acid activity, nucleic acid binding to metals, chromatography supports, glass, etc.

In the case of promoter variants, the variant sequences can be fused to reporter genes such as GFP or GUS. The variants can then be screened using fluorescence or or any other known technique for detection of enzymatic activity.

Genes Encoding Recombination Proteins

In the methods of the present invention, the filamentous fungal cell comprises a heterologous gene encoding a recombination protein. The gene encoding the recombination protein may be any isolated nucleic acid sequence encoding a recombination protein. The term “heterologous gene” is defined herein as a gene that encodes a recombination protein that is not native to the filamentous fungal cell; a native gene in which modifications have been made to alter the native sequence; or a native gene whose expression is quantitatively altered as a result of a manipulation by recombinant DNA techniques. For example, a native recombination protein may be recombinantly produced by, for example, placing a gene encoding the recombination protein under the control of a strong promoter.

The recombination protein promotes the recombination of the two or more regions of the DNA fragments with the corresponding homologous region in the DNA sequence to incorporate the DNA fragments therein by homologous recombination. In the methods of the present invention, any region that is homologous with the DNA sequence may be used.

In a preferred embodiment, the gene encoding the recombination protein is selected from the group consisting of: (a) a nucleic acid sequence having at least 70% identity with SEQ ID NO:2, SEQ ID NO:4 or SEQ ID NO:6. (b) a nucleic acid sequence having at least 70% homology with SEQ ID NO:1, SEQ ID NO:3 or SEQ ID NO:5; (c) a nucleic acid sequence which hybridizes under medium stringency conditions with (i) SEQ ID NO:1, SEQ ID NO:3, or SEQ ID NO:5, (ii) the cDNA sequence contained in SEQ ID NO:1, SEQ ID NO:3 or SEQ ID NO:5, or (iii) a complementary strand of (i) or (ii); (d) an allelic variant of (a), (b), or (c); and (e) a subsequence of (a), (b), (c), or (d), wherein the subsequence encodes a polypeptide fragment which has recombination activity.

In a first embodiment, the genes encoding recombination proteins have an amino acid sequence which have a degree of identity to SEQ ID NO:2, SEQ ID NO:4, or SEQ ID NO:6 of at least about 70%, preferably at least 75%, preferably at least about 80%, more preferably at least about 85%, even more preferably at least about 90%, most preferably at least about 95%, and even most preferably at least about 97% (hereinafter “homologous polypeptides”). In a preferred embodiment, the homologous recombination polypeptides have an amino acid sequence which differs by five amino acids, preferably by four amino acids, more preferably by three amino acids, even more preferably by two amino acids, and most preferably by one amino acid from SEQ ID NO:2, SEQ ID NO:4, or SEQ ID NO:6. For purposes of the present invention, the degree of identity between two amino acid sequences is determined by the Clustal method (Higgins, 1989, CABIOS 5: 151-153) using the LASERGENE™ MEGALIGN™ software (DNASTAR, Inc., Madison, Wis.) with an identity table and the following multiple alignment parameters: Gap penalty of 10 and gap length penalty of 10. Pair wise alignment parameters are Ktuple=1, gap penalty=3, windows=5, and diagonals=5.

Preferably, the gene encoding recombination proteins comprises the amino acid sequence of SEQ ID NO:2, SEQ ID NO:4, or SEQ ID NO:6; or an allelic variant thereof; or a fragment thereof that has recombination activity. In a more preferred embodiment, the gene encoding a recombination protein comprises the amino acid sequence of SEQ ID NO:2, SEQ ID NO:4, or SEQ ID NO:6. In another preferred embodiment, the gene encoding a recombination protein consists of the amino acid sequence of SEQ ID NO:2, SEQ ID NO:4, or SEQ ID NO:6; or an allelic variant thereof; or a fragment thereof, wherein the recombination protein fragment has recombination activity.

The present invention also encompasses genes which encode a recombination protein having the amino acid sequence of SEQ ID NO:2, SEQ ID NO:4, or SEQ ID NO:6, which differ from SEQ ID NO:1, SEQ ID NO:3, or SEQ ID NO:5, respectively, by virtue of the degeneracy of the genetic code. The present invention also relates to subsequences of SEQ ID NO:1, SEQ ID NO:3, or SEQ ID NO:5 which encode fragments of SEQ ID NO:2, SEQ ID NO:4, or SEQ ID NO:6, which have recombination activity.

A subsequence of SEQ ID NO:1, SEQ ID NO:3 or SEQ ID NO:5 is a nucleic acid sequence encompassed by SEQ ID NO:1, SEQ ID NO:3 or SEQ ID NO:5 except that one or more nucleotides from the 5′ and/or 3′ end have been deleted. Preferably, a subsequence of SEQ ID NO:1 contains at least 900 nucleotides, more preferably at least 945 nucleotides, and most preferably at least 990 nucleotides. Preferably, a subsequence of SEQ ID NO:3 contains at least 1500 nucleotides, more preferably at least 1560 nucleotides, and most preferably at least 1620 nucleotides. Preferably, a subsequence of SEQ ID NO:5 contains at least 2160 nucleotides, more preferably at least 2250 nucleotides, and most preferably at least 2350 nucleotides.

A fragment of SEQ ID NO:2, SEQ ID NO:4 or SEQ ID NO:6 is a protein having one or more amino acids deleted from the amino and/or carboxy terminus of this amino acid sequence.

Preferably, a fragment of SEQ ID NO:2 contains at least 300 amino acid residues, more preferably at least 315 amino acid residues, and most preferably at least 330 amino acid residues. Preferably, a fragment of SEQ ID NO:4 contains at least 500 amino acid residues, more preferably at least 520 amino acid residues, and most preferably at least 540 amino acid residues. Preferably, a fragment of SEQ ID NO:6 contains at least 720 amino acid residues, more preferably at least 750 amino acid residues, and most preferably at least 780 amino acid residues.

An allelic variant denotes any of two or more alternative forms of a gene occupying the same chromosomal locus. Allelic variation arises naturally through mutation, and may result in polymorphism within populations. Gene mutations can be silent (no change in the encoded recombination protein) or may encode recombination proteins having altered amino acid sequences. The allelic variant of a recombination protein is a recombination protein encoded by an allelic variant of a gene.

In a second embodiment, the genes encoding a recombination protein have a degree of homology to the recombination protein coding sequence of SEQ ID NO:1, SEQ ID NO:3 or SEQ ID NO:5 of at least about 70%, preferably at least about 75%, preferably at least about 80%, more preferably at least about 85%, even more preferably at least about 90%, most preferably at least about 95%, and even most preferably at least about 97% homology, which encode an active recombination protein; or allelic variants and subsequences of SEQ ID NO:1, SEQ ID NO:3 or SEQ ID NO:5 which encode recombination protein fragments which have recombination activity. For purposes of the present invention, the degree of homology between two nucleic acid sequences is determined by the Wilbur-Lipman method (Wilbur and Lipman, 1983, Proceedings of the National Academy of Science USA 80: 726-730) using the LASERGENE™ MEGALIGN™ software (DNASTAR, Inc., Madison, Wis.) with an identity table and the following multiple alignment parameters: Gap penalty of 10 and gap length penalty of 10. Pairwise alignment parameters are Ktuple=3, gap penalty=3, and windows=20.

In a third embodiment, the genes encoding recombination proteins hybridize under very low stringency conditions, preferably low stringency conditions, more preferably medium stringency conditions, more preferably medium-high stringency conditions, even more preferably high stringency conditions, and most preferably very high stringency conditions with a nucleic acid probe which hybridizes under the same conditions with (i) SEQ ID NO:1, SEQ ID NO:3, or SEQ ID NO:5, (ii) the cDNA sequence contained in SEQ ID NO:1, SEQ ID NO:3, or SEQ ID NO:5, (iii) a subsequence of (i) or (ii), or a complementary strand of (i), (ii), or (iii) (J. Sambrook, E. F. Fritsch, and T. Maniatus, 1989, Molecular Cloning, A Laboratory Manual, 2d edition, Cold Spring Harbor, N.Y.). The subsequence of SEQ ID NO:1, SEQ ID NO:3, or SEQ ID NO:5 may be at least 100 contiguous nucleotides or preferably at least 200 contiguous nucleotides. Moreover, the subsequence may encode a recombination protein fragment, which has recombination activity.

The nucleic acid sequence of SEQ ID NO:1, SEQ ID NO:3, or SEQ ID NO:5 or a subsequence thereof, as well as the amino acid sequence of SEQ ID NO:2, SEQ ID NO:4, or SEQ ID NO:6, or a fragment thereof, may be used to design a nucleic acid probe to identify and clone DNA encoding recombination proteins having recombination activity from strains of different genera or species according to methods well known in the art. In particular, such probes can be used for hybridization with the genomic or cDNA of the genus or species of interest, following standard Southern blotting procedures, in order to identify and isolate the corresponding gene therein. Such probes can be considerably shorter than the entire sequence, but should be at least 15, preferably at least 25, and more preferably at least 35 nucleotides in length. Longer probes can also be used. Both DNA and RNA probes can be used. The probes are typically labeled for detecting the corresponding gene (for example, with ³²P, ³H, ³⁵S, biotin, or avidin). Such probes are encompassed by the present invention.

Thus, a genomic DNA or cDNA library prepared from such other organisms may be screened for DNA, which hybridizes with the probes described above and which encodes a recombination protein having recombination activity. Genomic or other DNA from such other organisms may be separated by agarose or polyacrylamide gel electrophoresis, or other separation techniques. DNA from the libraries or the separated DNA may be transferred to and immobilized on nitrocellulose or other suitable carrier material. In order to identify a clone or DNA which is homologous with SEQ ID NO:1, SEQ ID NO:3, or SEQ ID NO:5; or a subsequence thereof, the carrier material is used in a Southern blot. For purposes of the present invention, hybridization indicates that the nucleic acid sequence hybridizes to a labeled nucleic acid probe corresponding to the nucleic acid sequence shown in SEQ ID NO:1, SEQ ID NO:3, or SEQ ID NO:5, its complementary strand, or a subsequence thereof, under very low to very high stringency conditions. Molecules to which the nucleic acid probe hybridizes under these conditions are detected using X-ray film.

In a preferred embodiment, the nucleic acid probe is a nucleic acid sequence which encodes the recombination protein of SEQ ID NO:2, SEQ ID NO:4, or SEQ ID NO:6; or a subsequence thereof. In another preferred embodiment, the nucleic acid probe is SEQ ID NO:1, SEQ ID NO:3, or SEQ ID NO:5. In another preferred embodiment, the probe is the nucleic acid sequence encoding a recombination protein contained in plasmid pZL1 rdhA13 that is contained in Escherichia coli NRRL B-30503. In another preferred embodiment, the probe is the nucleic acid sequence encoding the recombination protein contained in plasmid pZL1rdhB6 that is contained in Escherichia coli NRRL B-30503. In another preferred embodiment, the probe is the nucleic acid sequence encoding a recombination protein contained in plasmid pZL1 rdhD17 that is contained in Escherichia coli NRRL B-30505. In another preferred embodiment, the probe is the nucleic acid sequence encoding a recombination protein contained in plasmid pZL1rdhD10 that is contained in Escherichia coli NRRL B-30506.

For long probes of at least 100 nucleotides in length, very low to very high stringency conditions are defined as prehybridization and hybridization at 42° C. in 5×SSPE, 0.3% SDS, 200 μg/ml sheared and denatured salmon sperm DNA, and either 25% formamide for very low and low stringencies, 35% formamide for medium and medium-high stringencies, or 50% formamide for high and very high stringencies, following standard Southern blotting procedures.

For long probes of at least 100 nucleotides in length, the carrier material is finally washed three times each for 15 minutes using 2 x SSC, 0.2% SDS preferably at least at 45° C. (very low stringency), more preferably at least at 50° C. (low stringency), more preferably at least at 55° C. (medium stringency), more preferably at least at 60° C. (medium-high stringency), even more preferably at least at 65° C. (high stringency), and most preferably at least at 70° C. (very high stringency).

For short probes which are about 15 nucleotides to about 70 nucleotides in length, stringency conditions are defined as prehybridization, hybridization, and washing post-hybridization at 5° C. to 10° C. below the calculated Tₘ using the calculation according to Bolton and McCarthy (1962, Proceedings of the National Academy of Sciences USA 48:1390) in 0.9 M NaCl, 0.09 M Tris-HCl pH 7.6,6 mM EDTA, 0.5% NP-40, 1× Denhardt's solution, 1 mM sodium pyrophosphate, 1 mM sodium monobasic phosphate, 0.1 mM ATP, and 0.2 mg of yeast RNA per ml following standard Southern blotting procedures. For short probes which are about 15 nucleotides to about 70 nucleotides in length, the carrier material is washed once in 6×SCC plus 0.1% SDS for 15 minutes and twice each for 15 minutes using 6×SSC at 5° C. to 10° C. below the calculated Tₘ.

In a fourth embodiment, the genes encode variants of the recombination protein having an amino acid sequence of SEQ ID NO:2, SEQ ID NO:4, or SEQ ID NO:6, comprising a substitution, deletion, and/or insertion of one or more amino acids.

The amino acid sequences of the variant recombination proteins may differ from the amino acid sequence of SEQ ID NO:2, SEQ ID NO:4, or SEQ ID NO:6, by an insertion or deletion of one or more amino acid residues and/or the substitution of one or more amino acid residues by different amino acid residues. Preferably, amino acid changes are of a minor nature, that is conservative amino acid substitutions that do not significantly affect the folding and/or activity of the protein; small deletions, typically of one to about 30 amino acids; small amino- or carboxyl-terminal extensions, such as an amino-terminal methionine residue; a small linker peptide of up to about 20-25 residues; or a small extension that facilitates purification by changing net charge or another function, such as a poly-histidine tract, an antigenic epitope or a binding domain.

Examples of conservative substitutions are within the group of basic amino acids (arginine, lysine and histidine), acidic amino acids (glutamic acid and aspartic acid), polar amino acids (glutamine and asparagine), hydrophobic amino acids (leucine, isoleucine and valine), aromatic amino acids (phenylalanine, tryptophan and tyrosine), and small amino acids (glycine, alanine, serine, threonine and methionine). Amino acid substitutions which do not generally alter the specific activity are known in the art and are described, for example, by H. Neurath and R. L. Hill, 1979, In, The Proteins, Academic Press, New York. The most commonly occurring exchanges are Ala/Ser, Val/Ile, Asp/Glu, Thr/Ser, Ala/Gly, Ala/Thr, Ser/Asn, Ala/Val, Ser/Gly, Tyr/Phe, Ala/Pro, Lys/Arg, Asp/Asn, Leu/Ile, Leu/Val, Ala/Glu, and Asp/Gly as well as these in reverse.

The genes encoding recombination proteins may be obtained from microorganisms of any genus. For purposes of the present invention, the term “obtained from” as used herein in connection with a given source shall mean that the recombination protein encoded by the nucleic acid sequence is produced by the source or by a cell in which the nucleic acid sequence from the source has been inserted.

The genes encoding recombination proteins may be obtained from any filamentous fungal source including, but not limited to, an Acremonium, Aspergillus, Aureobasidium, Cryptococcus, Filibasidium, Fusarium, Humicola, Magnaporthe, Mucor, Myceliophthora, Neocallimastix, Neurospora, Paecilomyces, Penicillium, Piromyces, Schizophyllum, Talaromyces, Thermoascus, Thielavia, Tolypocladium, or Trichoderma strain.

In a preferred embodiment, the genes encoding recombination proteins are obtained from a Fusarium bactridioides, Fusarium cerealis, Fusarium crookwellense, Fusarium culmorum, Fusarium graminearum, Fusarium graminum, Fusarium heterosporum, Fusarium negundi, Fusarium oxysporum, Fusarium reticulatum, Fusarium roseum, Fusarium sambucinum, Fusarium sarcochroum, Fusarium sporotrichioides, Fusarium sulphureum, Fusarium torulosum, Fusarium trichothecioides, Fusarium venenatum, Humicola insolens, Humicola lanuginosa, Mucor miehei, Myceliophthora thermophila, Neurospora crassa, Penicillium purpurogenum, Trichoderma harzianum, Trichoderma koningii, Trichoderma longibrachiatum, Trichoderma reesei, or Trichoderma viride strain.

In another preferred embodiment, the genes encoding recombination proteins are obtained from an Aspergillus aculeatus, Aspergillus awamori, Aspergillus foetidus, Aspergillus japonicus, Aspergillus nidulans, Aspergillus niger, or Aspergillus oryzae strain.

In a more preferred embodiment, the genes encoding recombination proteins are obtained from Aspergillus oryzae.

It will be understood that for the aforementioned species, the invention encompasses both the perfect and imperfect states, and other taxonomic equivalents, e.g., anamorphs, regardless of the species name by which they are known. Those skilled in the art will readily recognize the identity of appropriate equivalents.

Strains of these species are readily accessible to the public in a number of culture collections, such as the American Type Culture Collection (ATCC), Deutsche Sammlung von Mikroorganismen und Zellkulturen GmbH (DSM), Centraalbureau Voor Schimmelcultures (CBS), and Agricultural Research Service Patent Culture Collection, Northern Regional Research Center (NRRL).

Furthermore, such genes encoding recombination proteins may be identified and obtained from other sources including microorganisms isolated from nature (e.g., soil, composts, water, etc.) using the above-mentioned probes. Techniques for isolating microorganisms from natural habitats are well known in the art. The gene may then be derived by similarly screening a genomic or cDNA library of another microorganism. Once a gene encoding a polypeptide has been detected with the probe(s), the sequence may be isolated or cloned by utilizing techniques which are known to those of ordinary skill in the art (see, e.g., Sambrook et al., 1989, supra).

In a most preferred embodiment, the gene encoding the recombination protein is set forth in SEQ ID NO:1. In another most preferred embodiment, the gene is the sequence contained in plasmid pZL1rdhA13 that is contained in Escherichia coli NRRL B-30503. In another most preferred embodiment, the gene is set forth in SEQ ID NO:3. In another most preferred preferred embodiment, the gene is the sequence contained in plasmid pZL1rdhB6 that is contained in Escherichia coli NRRL B-30503. In another most preferred embodiment, the gene is set forth in SEQ ID NO:5. In another most preferred preferred embodiment, the gene is the sequence contained in plasmid pZL1rdhD17 that is contained in Escherichia coli NRRL B-30505. In another most preferred embodiment, the gene is set forth in SEQ ID NO:7. In another most preferred embodiment, the gene is the sequence contained in plasmid pZL1rdhD10 that is contained in Escherichia coli NRRL B-30506.

The present invention also relates to mutant genes encoding recombination proteins comprising at least one mutation in the recombination protein coding sequence of SEQ ID NO:1, SEQ ID NO:3, or SEQ ID NO:5 in which the mutant gene encodes a polypeptide which consists of SEQ ID NO:2, SEQ ID NO:4, or SEQ ID NO:6, respectively.

The techniques used to isolate or clone a gene are known in the art and include isolation from genomic DNA, preparation from cDNA, or a combination thereof. The cloning of the genes from such genomic DNA can be effected, e.g., by using the well-known polymerase chain reaction (PCR) or antibody screening of expression libraries to detect cloned DNA fragments with shared structural features. See, e.g., Innis et al., 1990, PCR: A Guide to Methods and Application, Academic Press, New York. Other nucleic acid amplification procedures such as ligase chain reaction (LCR), ligated activated transcription (LAT) and nucleic acid sequence-based amplification (NASBA) may be used.

In the methods of the present invention, the genes encoding recombination proteins are preferably overexpressed. Overexpression of these genes can be accomplished by multiple insertions of the genes in the genome of the filamentous fungal host cell and/or by substituting heterologous control sequences for the native control sequences in the gene, e.g., a strong promoter.

The present invention is further described by the following examples which should not be 15 construed as limiting the scope of the invention.

### EXAMPLES

### Example 1

### Aspergillus oryzae Genomic DNA Extraction

Aspergillus oryzae HowB101, Aspergillus oryzae HowB430, or Aspergillus oryzae HowB425 was grown in 25 ml of 0.5% yeast extract-2% glucose (YEG) medium for 24 hours at 37° C. and 250 rpm. Mycelia were then collected by filtration through Miracloth (Calbiochem, La Jolla, Calif.) and washed once with 25 ml of 10 mM Tris-1 mM EDTA (TE) buffer. Excess buffer was drained from the mycelia preparation which was subsequently frozen in liquid nitrogen. The frozen mycelia preparation was ground to a fine powder in an electric coffee grinder, and the powder was added to a disposable plastic centrifuge tube containing 20 ml of TE buffer and 5 ml of 20% w/v sodium dodecylsulfate (SDS). The mixture was gently inverted several times to ensure mixing, and extracted twice with an equal volume of phenol:chloroform:isoamyl alcohol (25:24:1 v/v/v). Sodium acetate (3 M solution) was added to the extracted sample to a final concentration of 0.3 M followed by 2.5 volumes of ice cold ethanol to precipitate the DNA. The tube was centrifuged at 15,000×g for 30 minutes to pellet the DNA. The DNA pellet was allowed to air-dry for 30 minutes before resuspension in 0.5 ml of TE buffer. DNase-free ribonuclease A was added to the resuspended DNA pellet to a concentration of 100 μg per ml and the mixture was then incubated at 37° C. for 30 minutes. Proteinase K (200 μg/ml) was added and the tube was incubated an additional one hour at 37° C. Finally, the sample was extracted twice with phenol:chloroform:isoamyl alcohol and the DNA precipitated with ethanol. The precipitated DNA was washed with 70% ethanol, dried under vacuum, resuspended in TE buffer, and stored at 4° C.

### Example 2

### PCR Amplification of a Portion of the Aspergillus oryzae rdhA Gene

A portion of the Aspergillus oryzae rdhA (rad51 homolog A) gene was amplified by hemi-nested degenerate PCR. The first amplification employed degenerate primers 971514 and 971515, shown below, coding for amino acids DNVAYAR and MFNPDPK. Primer 971514 (DNVAYAR): 5′-GAYMYGTIGCITAYGCNMG-3′ (SEQ ID NO:7) Primer 971515 (MFNPDPK): 5′-TTIGGRTCNGGRTTRAACAT-3′ (SEQ ID NO:8)

The amplification reactions (30 pi) were prepared using Aspergillus oryzae HB101 genomic DNA as template with the following components: PCR buffer II (Perkin Elmer, Branchburg, N.J.),0.25 mM dNTPs, 0.8 μg of Aspergillus oryzae HowB101 genomic DNA, 6.4 μM primer 971514, 3.2 μM primer 971515, and 1.5 units of Taq DNA polymerase (Perkin Elmer, Branchburg, N.J.). Before amplification, the template DNA was denatured in a boiling water bath for 5 minutes and quick-cooled on ice. The reaction was initiated by adding Taq DNA polymerase to the other reaction components at 72° C. The reactions were incubated in a Perkin-Elmer Model 480 Thermal Cycler programmed as follows: 35 cycles each for 20 seconds at 94° C., 30 seconds at 66° C., 60 seconds ramping from 66 to 50° C., and 60 seconds at 72° C. (5 minute final extension). The reaction products were isolated on a 1.6% agarose gel using 40 mM Tris base-20 mM sodium acetate-1 mM disodium EDTA (TAE) buffer where a 300 bp product band was excised from the gel and purified using a QIAquick Gel Extraction Kit (QIAGEN, Chatsworth, Calif.) according to the manufacturer's instructions.

One-tenth of the isolated 300 bp product was amplified under the same conditions described above except that primer 971516, shown below, was used in place of primer 971515. Primer 971516 (NQWAQV): 5′-ACYTGIGCIACNACYTGRTT-3′ (SEQ ID NO:9) The products were fractionated as before and a band at approximately 260 bp was excised and purified as described for the 300 bp product.

The purified PCR product was subsequently subcloned using the TOPO TA Cloning kit (Invitrogen, Carlsbad, Calif.) according to the manufacturer's instructions and the DNA sequence was determined using M13 Forward (−20) Primer (Invitrogen, Carlsbad, Calif.). DNA sequence analysis of the 260 bp rdhA gene segment showed that the amplified gene segment encoded a portion of the corresponding Aspergillus oryzae rdhA gene.

### Example 3

### Isolation of a Full-Length Aspergillus oryzae rdhA Genomic Clones

Genomic DNA libraries were constructed using the bacteriophage cloning vector λZipLox (Life Technologies, Gaithersburg, Md.) with E. coli Y1090ZL cells (Life Technologies, Gaithersburg, Md.) as a host for plating and purification of recombinant bacteriophage and E. coli DH10Bzip (Life Technologies, Gaithersburg, Md.) for excision of individual pZL1 clones containing the rdhA gene.

Aspergillus oryzae HowB425 genomic DNA was partially digested with Tsp5091 and size-fractionated on 1% agarose gels. DNA fragments migrating in the size range 3-7 kb were excised and eluted from the gel using Prep-a-Gene reagents (BioRad Laboratories, Hercules, Calif.). The eluted DNA fragments were ligated with EcoRI-cleaved and dephosphorylated λZipLox vector arms (Life Technologies, Gaithersburg, Md.), and the ligation mixtures were packaged using commercial packaging extracts (Stratagene, La Jolla, Calif.). The packaged DNA libraries were plated and amplified in Escherichia coli Y1090ZL cells (Life Technologies, Gaithersburg, Md.).

The Aspergillus oryzae HowB425 DNA library was plated on NZCYM agar plates. Plaque lifts (Maniatis et al., 1982, Molecular Cloning, A Laboratory Manual, Cold Spring Harbor Press, Cold Spring Harbor, N.Y.) were performed on approximately 40,000 pfu and the DNA was fixed onto membranes by heating at 80° C. for two hours. The membranes were soaked for 30 minutes at 65° C. in a hybridization solution containing 6×SSPE and 7.0% SDS.

The subcloned rdhA product of the PCR amplification described in Example 2 was excised from the vector pCR2.1-TOPO by digestion with EcoRI. Approximately 28 ng was random-primer labeled using a Stratagene Prime-It II Kit (Stratagene, La Jolla, Calif.) according to the manufacturer's instructions and used to probe the approximately 40,000 pfu of the Aspergillus oryzae genomic library constructed from Aspergillus oryzae strain HowB425 in the vector λZipLox. The radiolabeled rdhA gene fragment was then denatured by adding sodium hydroxide to a final concentration of 0.5 M, and added to the hybridization solution at an activity of approximately 1×10⁶ cpm per ml of hybridization solution. The mixture was incubated overnight at 65° C. in a shaking water bath. Following incubation, the membranes were washed two times in 0.2×SSC with 0.2% SDS at room temperature and an additional two times in the same solution at 65° C. The membranes were then sandwiched between sheets of plastic and exposed to X-ray film for 18 hours at −80° C. with intensifying screens (Kodak, Rochester, N.Y.).

Fourteen plaques produced strong hybridization signals with the probe. Twelve of the plaques were picked from the plates and eluted overnight in 1 ml of SM (5.8 g/l NaCl, 2 g/l MgSO₄.7H₂O, 50 mM Tris-Cl, 0.01% gelatin). For plaque purification, the eluates were diluted 1:100 and 2 μl of the dilution was plated on NZCYM plates together with Y1090ZL plating bacteria. Plaque lifts were prepared and screened as described above, and individual plaques were picked into 0.5 ml of SM. The pZL1 plasmids were excised from the purified phagemid clones according to the protocol suggested by Life Technologies (Gaithersburg, Md.). Colonies were inoculated into three ml of LB plus 50 μg/ml ampicillin medium and grown overnight at 37° C. Miniprep DNA was prepared from each of these clones using the Qiagen Bio Robot 9600 according to the manufacturer's protocol. The plasmids were digested with EcoRI and XbaI and fractionated by agarose gel electrophoresis in order to determine if the clones were identical and to determine their sizes. The nine unique clones had insert sizes ranging from 3.15 to 6.4 kb.

### Example 4

### Characterization of the Aspergillus oryzae Genomic Clone Encoding RDHA

DNA Sequencing of Each Clone was Performed with an Applied Biosystems Prism 377

DNA Sequencer using the BigDye Terminator Cycle Sequencing Ready Reaction kit (ABI, Foster City, Calif.) according to the manufacturer's instructions. Oligonucleotide sequencing primers were designed to complementary sequences in the pZL1 plasmid vector and were synthesized by Operon Technologies Inc., Alameda, Calif. Contig sequences were generated by sequencing from the ends of each pZL1 clone and by sequencing subclones obtained from SalI, PstI, or HindIII digests of Clone #3, Clone #7, Clone #12, or Clone 13.

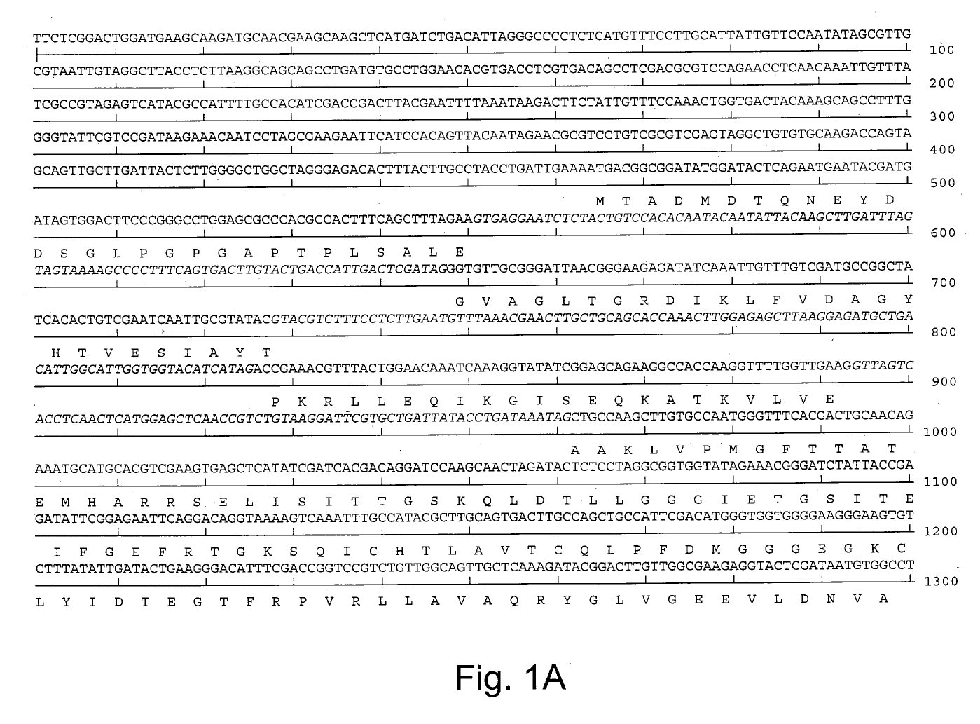

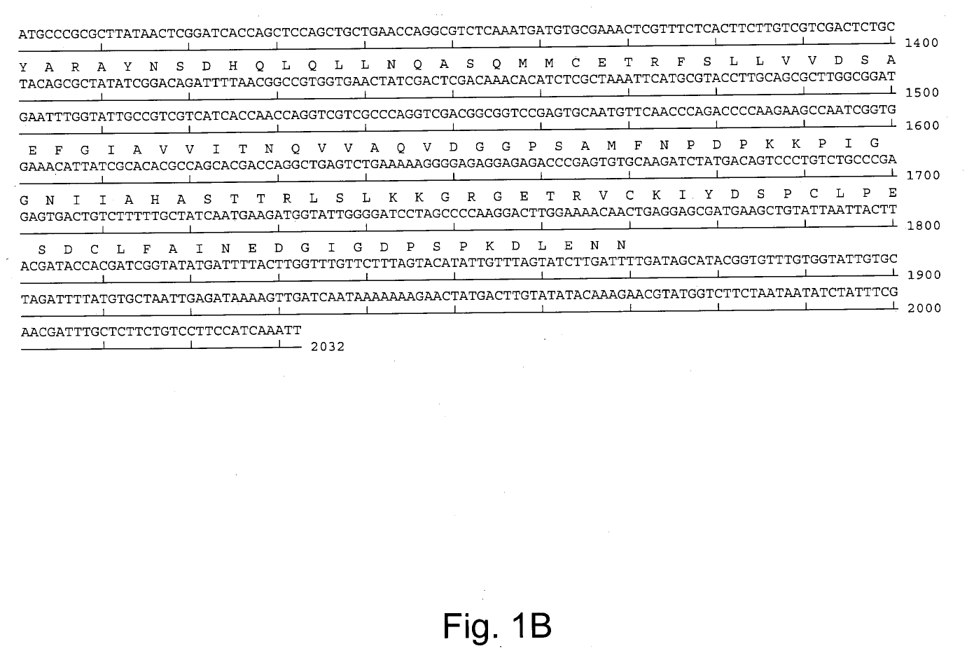

The 1.3 kb genomic region encompassing the coding sequence was sequenced to an average redundancy of 5.9. The nucleotide sequence and deduced amino acid sequence are shown in FIG. 1 (SEQ ID NOs: 1 and 2). Sequence analysis of the cloned insert revealed a coding sequence of 1307 bp (excluding the stop codon) encoding a protein of 348 amino acids. The coding sequence is punctuated by three introns of 97 bp, 98 bp, and 68 bp. The G+C content of the coding sequence is 55.3%. The predicted RDHA polypeptide has a molecular mass of 37.6 kdal and an isoelectric point of 5.24. Using the Signal P software program (Nielsen et al., 1997, Protein Engineering 10: 1-6), no signal peptide was predicted (Y<0.027).

A comparative alignment of the Aspergillus oryzae RDHA protein sequence with other sequences using the Clustal W algorithm in the Megalign program of DNASTAR, showed that the deduced amino acid sequence of the Aspergillus oryzae RDHA protein shares 98% identity to the deduced amino acid sequence of the UVSC protein of Emericella nidulans (accession number CAB02454).

Clone 13 was deposited as E. coli pZL1 rdhA13 (NRRL B-30503) on Jul. 27, 2001, with the Agricultural Research Service Patent Culture Collection, Northern Regional Research Center, 1815 University Street, Peoria, Ill.

### Example 5

### PCR Amplification of a portion of the Aspergillus oryzae rdhb gene

A portion of two Aspergillus oryzae genes homologous to the yeast rad52 gene were amplified by consensus/degenerate PCR (Rose et al., 1998, Nucleic Acids Res. 26: 1628-35). The amplification employed primers 980539 and 980540 shown below.

Primer 980539 (ANEVFGFNGW):

5′-CGMCGMGTCTTCGGTTTYMYGGNTGG-3′ (SEQ ID NO:10)

Primer 980540 (KKEGTTDGMK):

5′-CTTCATGCCGTCGGTAGTNCCYTCYTTYTT-3′ (SEQ ID NO: 11)

The amplification reaction (30 pi) was prepared using Aspergillus oryzae HB425 genomic DNA as template with the following components: PCR buffer II (Perkin Elmer), 0.20 mM dNTPs, 0.4 μg of Aspergillus oryzae HowB425 genomic DNA, 5.0 μM primer 980539, 5.0 μM primer 980540, and 3.0 units of Taq DNA polymerase. Before amplification, the template DNA was denatured in a boiling water bath for 5 minutes and quick-cooled on ice. The reaction was initiated by adding Taq DNA polymerase to the other reaction components at 72° C. The reactions were incubated in a Stratagene Robocycler programmed for 35 cycles each for 30 seconds at 94° C., 60 seconds at 53° C., and 90 seconds at 72° C. (7 minutes final extension).

The amplification products were fractionated as described above for the rdhA gene, and bands at about 350 and 300 bp were excised and cloned using the TOPO TA cloning kit according to the manufacturer's instructions and the DNA sequence was determined using T7 promoter primer. DNA sequence analysis of the 350 and 300 bp gene segments showed that the amplified gene segments encoded a portion of two closely related Aspergillus oryzae genes, hereafter designated as rdhB (rad52 homolog B) and rdhC (rad52 homolog C), respectively.

### Example 6

### Isolation of a Full-Length Aspergillus oryzae rdhb Genomic Clone

Approximately 50 ng of the gel-purified ca. 300-bp product of the PCR amplification described in Example 3 was random-primer labeled using a Stratagene Prime-It II Kit according to the manufacturer's instructions and used to probe approximately 100,000 pfu of an Aspergillus oryzae genomic library constructed from Aspergillus oryzae strain HowB430 in the vector λZipLox using the same procedures described in Example 3.

Eleven hybridizing plaques were obtained, and four of these were purified, excised as pZL1 clones, and characterized as described in Example 3. The two unique clones obtained had insert sizes of approximately 3.9 kb and 6.3 kb. The larger clone was designated E. coli pZL1 clone #6 and submitted to sequence analysis (see Example 7).

### Example 7

### Characterization of the Aspergillus oryzae Genomic Clone Encoding RDHB

DNA sequencing of each clone was performed with an Applied Biosystems Prism 377 DNA Sequencer using the BigDye Terminator Cycle Sequencing Ready Reaction kit according to the manufacturer's instructions. Oligonucleotide sequencing primers were designed to complementary sequences in the pZL1 plasmid vector and were synthesized by Operon Technologies Inc., Alameda, Calif. Contig sequences were generated using a transposon insertion strategy (Primer Island Transposition Kit, Perkin-Elmer/Applied Biosystems, Inc., Foster City, Calif.).

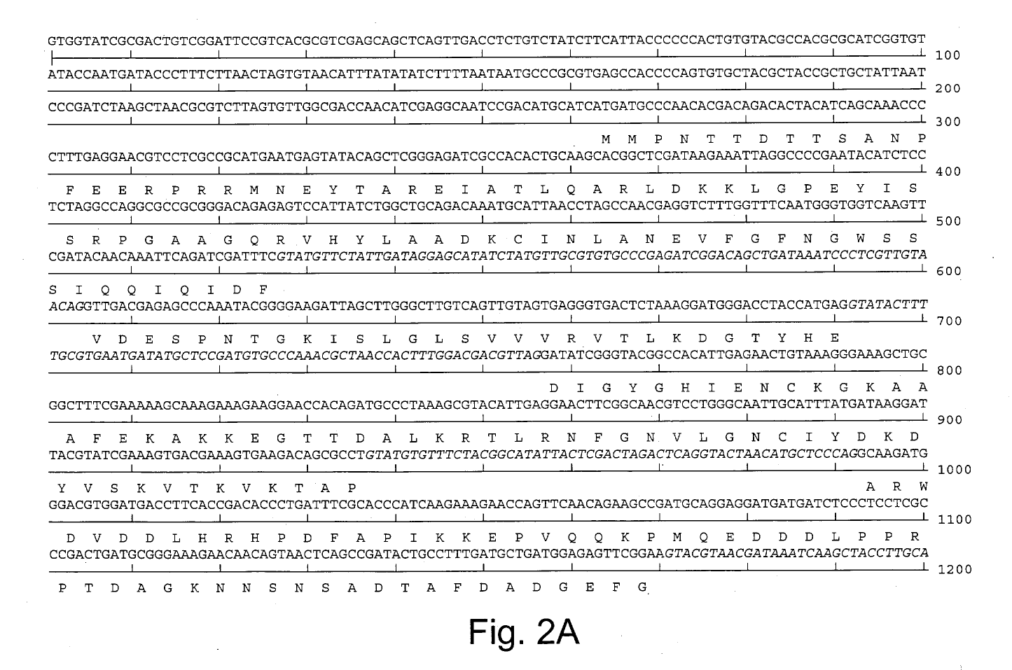

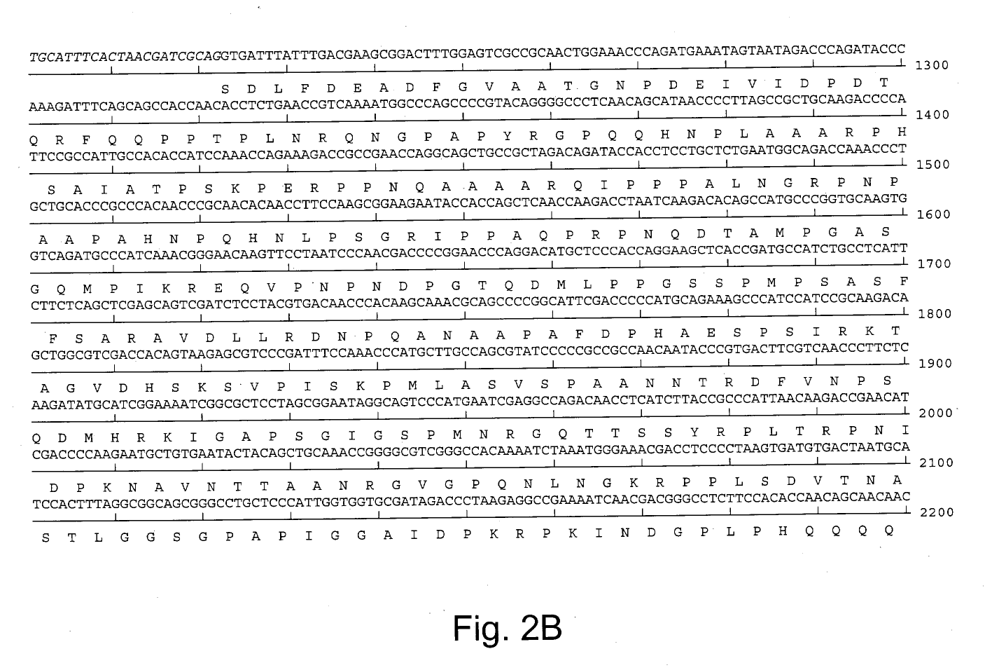

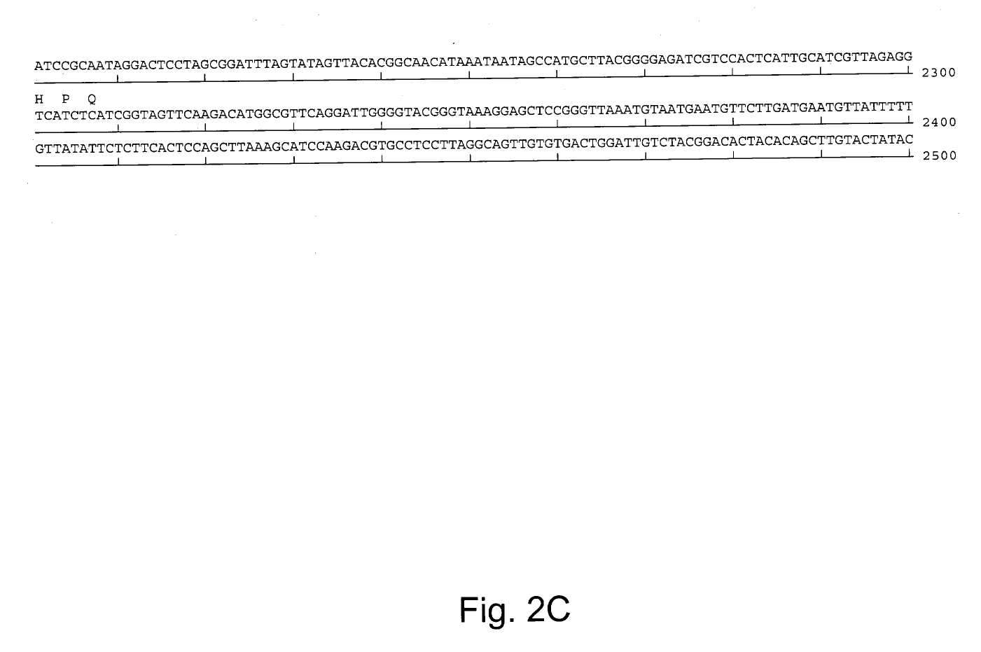

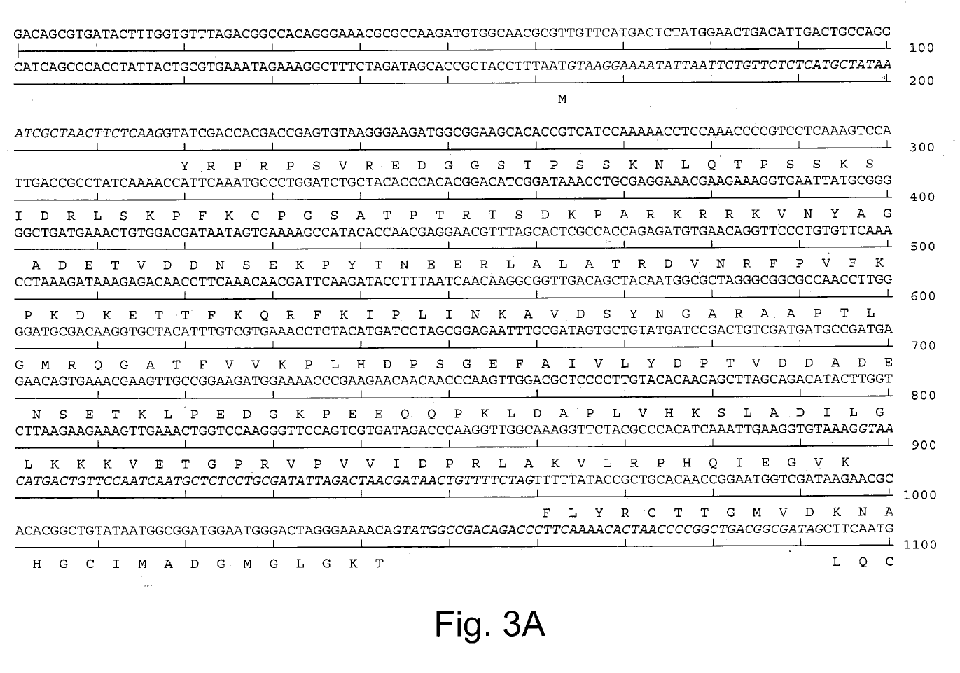

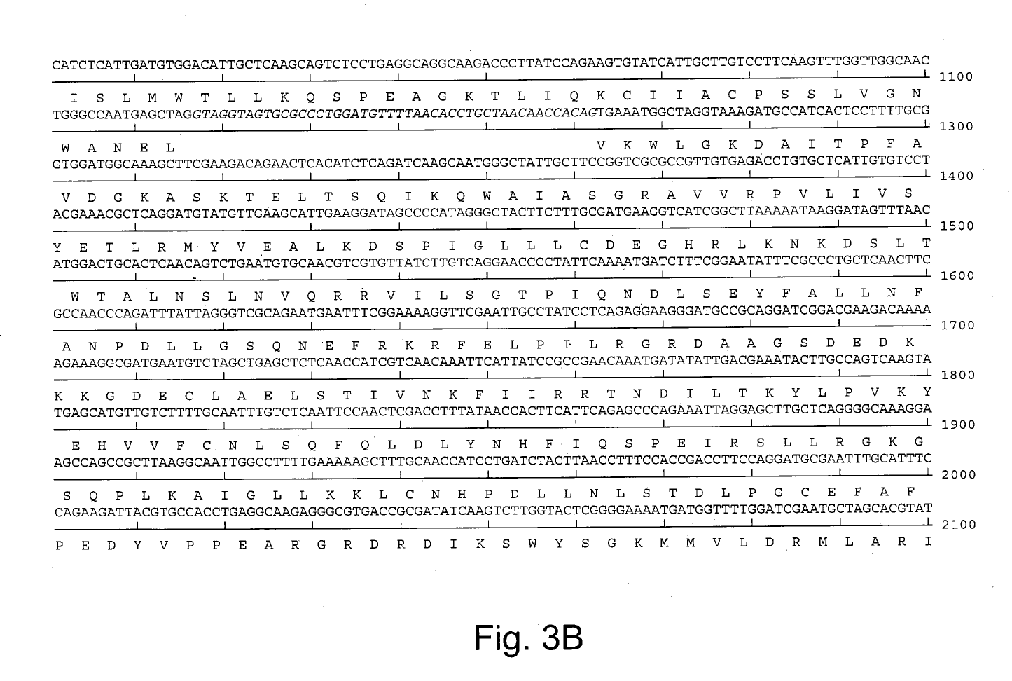

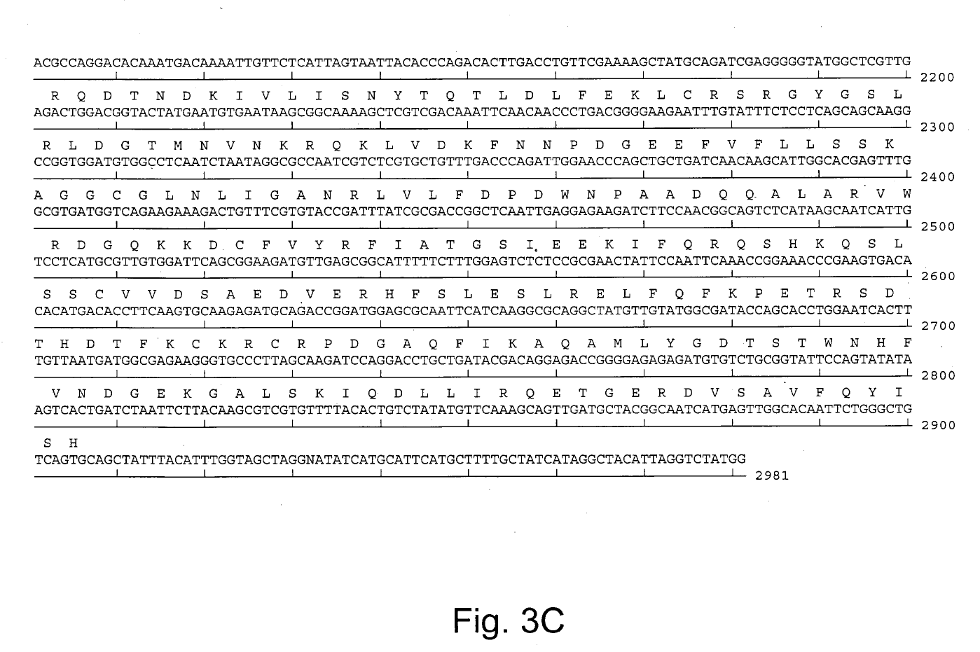

A 3257 bp genomic fragment was sequenced to an average redundancy of 4.7. The nucleotide sequence and deduced amino acid sequence are shown in FIG. 2 (SEQ ID NOs:3 and 4). Sequence analysis of the cloned insert revealed a coding sequence of 1946 bp (excluding the stop codon) encoding a protein of 565 amino acids. The coding sequence is punctuated by four introns of 78 bp, 65 bp, 56, and 52 bp. The G+C content of the coding sequence is 51.8%. The predicted RDHB polypeptide has a molecular mass of 60.7 kdal and an isoelectric point of 8.64. Using the Signal P software program (Nielsen et al., 1997, Protein Engineering 10: 1-6), no signal peptide was predicted (Y<0.043).

A comparative alignment of the Aspergillus oryzae RDHB protein sequence with other sequences using the Clustal W algorithm in the Megalign program of DNASTAR, showed that the deduced amino acid sequence of the Aspergillus oryzae RDHB protein shares 33% identity to the deduced amino acid sequence of the RAD22 protein of Schizosaccharomyces pombe (accession number P36592) and 33% identity to the RAD52 protein of Saccharomyces cerevisiae (accession number P06778).

Clone #6 was deposited as E. coli pZL1rdhB6 (NRRL B-30504) on Jul. 27, 2001, with the Agricultural Research Service Patent Culture Collection, Northern Regional Research Center, 1815 University Street, Peoria, Ill.

### Example 8

### Construction of pRamB33

Intermediates pRaMB31 and pRaMB32 were constructed as follows: First, plasmid pUC19 was digested with NdeI plus PvuII and the 2241 bp vector fragment, purfied by agarose gel electrophoresis, was ligated with the following synthetic linker which contains restriction sites for MunI, PacI, BamHI, HindIII, PmeI, and MunI while inactivating the NdeI cloning site:

5′-TATCMTTCTTMTTMGGATCCMGCTTGTTTAAACMTTC-3′ (SEQ ID NO:12)

3′-AGTTMCAATTMTTCCTAGGTTCGMCAAATTTGTTMC-5′-(SEQ ID NO:13)

The resulting pUC 19-derivative was termed pRaMB31. Next, the Aspergillus oryzae pgk promoter and terminator regions (Genbank accession number D28484) as well as the bargene from Streptomyces hygroscopicus (White et al. 1990, Nucleic Acids Res. 18: 1062) were amplified by PCR using the following primer pairs:

Aspergillus oryzae pgk promoter:

5′-GATACATGTTATGGAGATGTTCTATCACACMG-3′ (contains AfIII site) (SEQ ID NO:14)

5′-CAGGATCCTGCAGTATTGACTACTATGGT-3′ (contains BamHI site) (SEQ ID NO:15)

Aspergillus oryzae pgk terminator

5′-CTGTTTAAACTGCAGGGAGGAACTGAAAAAGG-3′ (contains PmeI site) (SEQ ID NO:16)

5′-GTTMGCTTGCGAAACGCAAATAATGTGTTG-3′ (contains HindIII site) (SEQ ID NO:17) Streptomyces hygroscopicus bar gene

5′-GTTACATGTCTCCAGAACGACGCCCGGCGGACATC-3′ (contains AfIII site) (SEQ ID NO:18)

5′-TGMGCTTCAGATCTCGGTGACGGGCAG-3′ (contains HindIII site) (SEQ ID NO:19)

The amplification reactions (100 μl) was prepared using pMT1612 (which harbors the bar gene from Streptomyces hygroscopicus—EMBL accession number X05822) as template with the following components: 1× Pwo buffer (Roche Molecular Biochemicals, Indianapolis, Ind.), 0.25 mM dNTPs, 1.0 μM of each primer, and 5 units of Pwo DNA polymerase. The reactions were incubated in an Applied Biosystems thermocycler programmed for 1 cycle at 95° C. for 3 minutes, 45° C. for 2 minutes, and 67° C. for 5 minutes followed by 30 cycles each at 95° C. for 2 minutes; 45° C. for 2 minutes; and 67° C. for 2 minutes.

The PCR-amplified pgk terminator was digested with HindIII plus PmeI and the 635 bp product was purified by agarose gel electrophoresis, then ligated with pRaMB31 that had been cleaved with the same enzymes. The resulting intermediate plasmid was designated as pRaMB31.1. Next, the pgk promoter and bar gene segments were digested with BamHI plus AfIII and HindII plus AfIII, respectively, and purified byelectrophoresis. These two fragments were combined in a three-part ligation with the intermediate pRaMB31.1 that had been digested with BamHI plus HindIII. The product of this ligation, pRaMB32 contained the Streptomyces hygroscopicus bargene under transcriptional control of the Aspergillus oryzae pgk promoter and terminator regions.

Next, the Aspergillus oryzae niaA promoter and alkaline protease (alp) terminator regions were amplified by PCR using high-fidelity Pwo polymerase (Boehringer-Mannheim) as above with the following primer pairs:

Aspergillus oryzae niaA promoter

5′-GGTTMTTAACCGGCAGGGMGGCCMTGAAAG-3′ (contains AfIII site) (SEQ ID NO:20)

5′-CCACGCGTATTTAAATGTCCGGGATGGATAGCACTGTGG-3′ (contains PacI site) (SEQ ID NO:21)

Aspergillus oryzae alp terminator

5′-GGACGCGTGCGGCCGCGTACCAGGAGTACGTCGCAGG-3′ (contains MluI site) (SEQ ID NO:22)

5′-GGAGATCTGCAGCTGTGTACCMTAGAC-3′ (contains BgIII site) (SEQ ID NO:23)

The amplified niaA promoter segment was cloned directly into pUC118 (Yanisch-Perron et al., 1985, Gene 33: 103-119), which had been digested with SmaI and dephosphorylated. Similarly, the alp terminator region was subcloned into pCR-blunt (Invitrogen, Carlsbad, Calif.). The nucleotide sequences of both products were determined to ensure accuracy. The niaA promoter fragment was isolated by gel electrophoresis following cleavage with PacI plus MluI, and the alp terminator segment was purified after digestion with MluI plus BglII. These purified fragments were mixed in a three-part ligation with pRaMB32 which had been previously cut with BamHI plus PacI. The resulting vector, designated as pRaMB33, contained (a) a selectable bar gene under the transcriptional control of the pgk promoter and terminator, and (b) unique NotI and SwaI restriction sites located between the niaA promoter and alp terminator for directional cloning of cDNA or other coding regions of interest.

### Example 9

### Construction of Expression Vector with niaA Promoter

Plasmid pRaMB33 was digested with XbaI and NruI to remove the Basta-resistance cassette. The remaining vector was isolated on a 0.8% agarose gel using TAE buffer where a 4.4 kb band was excised from the gel and purified using a QIAquick Gel Extraction Kit (QIAGEN, Chatsworth, Calif.) according to the manufacturer's instructions.

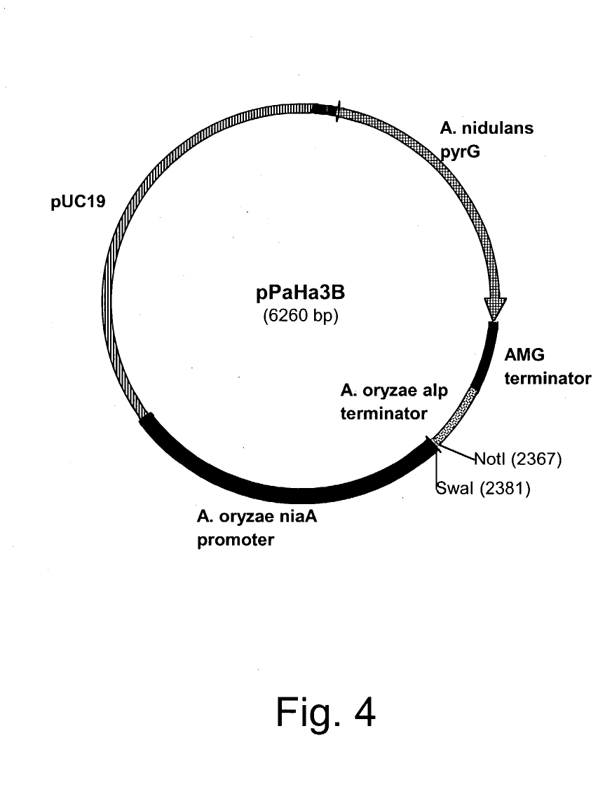

Plasmid pBANe13 (WO 97/47746) was digested with PmeI and NheI, and the fragment containing the pyrG gene and AMG terminator was similarly gel isolated and purified. The fragments were mixed together, blunt-ended using Klenow polymerase, ligated, and transformed into E. coli DH5a. Plasmid DNA was prepared from ten of the resulting transformants, and one displaying the correct restriction digest pattern was designated pPaHa3B (FIG. 4). The niaA promoter is induced by nitrate.

### Example 10

### Plasmids for Inter-Plasmid Recombination Assay

Plasmid pSMO122 (U.S. Pat. No. 5,958,727) was digested with HindIII and treated with bacterial alkaline phosphatase. Plasmid Arp1 (Gems et al., 1991, Gene 98: 61-67) was digested with HindIII and the digest fractionated on a 1.0% agarose gel in TAE buffer. A 5.8 kb fragment was excised from the gel and purified using a QIAquick Gel Extraction Kit (QIAGEN, Chatsworth, Calif.) according to the manufacturer's instructions. This fragment was ligated to the linearized pSMO122 plasmid and transformed into Escherichia coli DH5a. Plasmid DNA was prepared from transformants, and one, showing the correct fragment sizes after digestion with HindIII, was designated pHB217. The fragment contains the AMAL replication region from Emericella nidulans and the pyrG gene from Aspergillus oryzae.

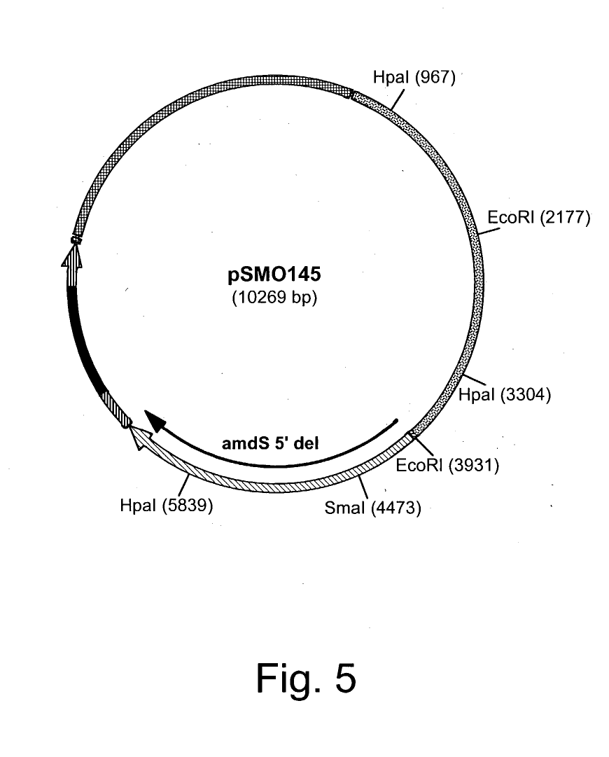

Plasmid pPaHa1-1 was digested NsiI and the ends were made blunt using T4 DNA polymerase. The products were fractionated on a 0.8% agarose gel using TAE buffer and a 2 kb band was excised from the gel and purified using a QIAEX Gel Extraction Kit (QIAGEN, Chatsworth, Calif.) according to the manufacturer's instructions. The fragment was then inserted into the SmaI site of pHB217. The plasmid was designated pSMO145 (FIG. 5). The plasmid carries a 220 bp deletion of the Emericella nidulans amds gene encompassing a portion of that gene's promoter, all of the 5′-untranslated region, and 132 bp of the coding region.

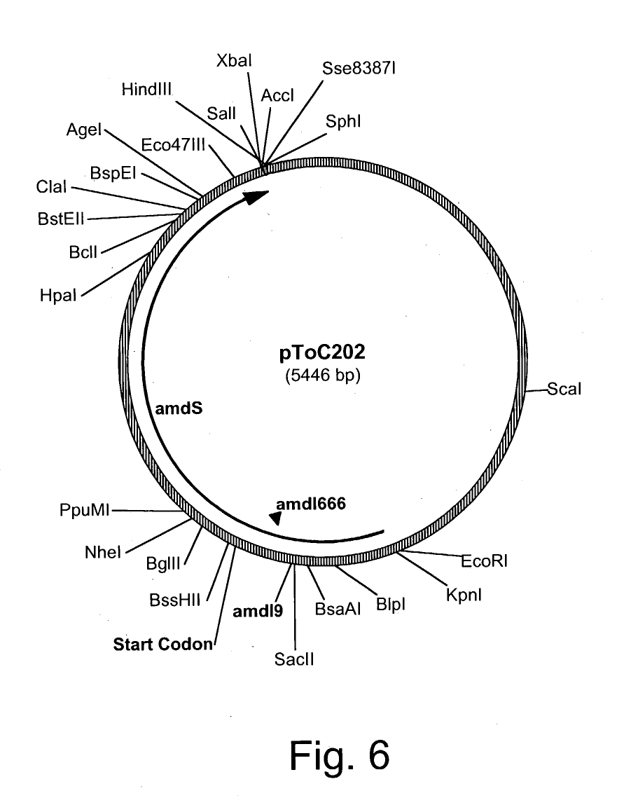

Plasmid pToC202 (FIG. 6) was constructed to contain three up promoter mutations have identified within the Aspergillus nidulans amds gene: The 1666 and 166 up mutations have been described by Katz et al., 1990, Mol. Gen. Genet. 220: 373-376. The 19 mutation has been described by Davis and Hynes, 1989, TIG 5:14-19 and by Todd, 1998, EMBO 17: 2042-2054. Plasmid pI66PI9 contains the Aspergillus nidulans amds with the two up promoter mutations 166 and I9. The amds allele of this plasmid was subcloned into pUCI9 as a 2,7 kb XbaI fragment to form the plasmid pToC186C. (Yanisch-Perron et al., 1985, Gene 33 103-119).

Plasmid pMSX-6B1 contains the Aspergillus nidulans amds gene with the up promoter mutation 1666. The amds allele of this plasmid was subcloned into pUC19 as a 2.7 kb Xbal fragment to form the plasmid pToC196. The 19 and 1666 mutations were combined by inserting a 544 bp XmaI fragment from pToC186 harboring the 19 mutation into the 4903 bp XmaI fragment of pToC196 to form the plasmid pToC202 (FIG. 6).

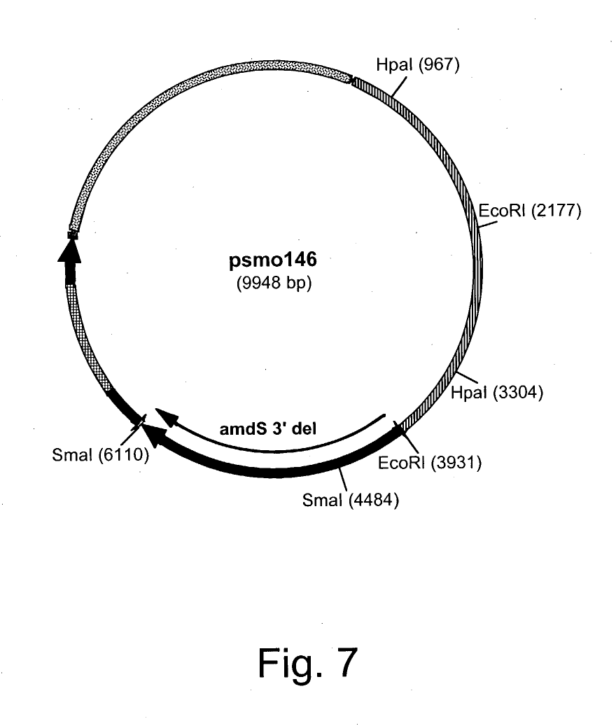

A 3′ truncation of the Emericella nidulans amds gene was produced by digesting plasmid pToC202 with EcoRI and HpaI, blunting with Klenow fragment, gel and purified using a QIAEX Gel Extraction Kit according to the manufacturer's instructions. The fragment was then inserted into the SmaI site of pHB217. The resulting plasmid was designated pSMO146 (FIG. 7). The promoter region of amds in this construct contained mutations that enhance promoter strength, allowing good growth on acetamide as the sole nitrogen source with a single copy of the gene.

### Example 11

### Construction of Aspergillus oryzae rdhA and rdhB Overexpression Vectors

Plasmid pRaMB32 (described in Example 8) was digested with PstI and ScaI and fractionated on a 1% agarose gel. The 2.8 kb band containing the pgk promoter, bargene, and pgk terminator was excised and purifed with the Qiagen QIAEX II kit (QIAGEN, Chatsworth, Calif.) according to the manufacturer's instructions. Plasmid pBANe8 (U.S. Pat. No. 5,958,727) was digested with NsiI and dephosphorylated using 150 units of bacterial alkaline phosphatase followed by heat inactivation at 65° C. for 1 hour. The digest was fractionated on a 1% agarose gel and the 5.0 kb band was excised and purified as above. The two fragments were ligated together and transformed into E. coli XL10 Gold cells (Stratagene, La Jolla, Calif.) according to the manufacturer's instructions. Plasmid DNA was prepared from transformants and screened for correctness by digesting with StuI. One plasmid showing the correct digestion pattern was named pBANe44.

The 1.3 kb coding region of the Aspergillus oryzae rdhA gene was amplified by PCR from E. coli pZL1 clone #13. Primers incorporated SwaI, PacI, or NotI sites for subsequent cloning and had the following sequence:

Sense Swa primer (980442):

5′-CATTTAAATGATGACGGCGGATATG-3′ (SEQ ID NO:24)

Antisense Pac primer (980359):

5′-GTTMTTMTCAGTTGTTTTCCAAGTC-3′ (SEQ ID NO:25)

Antisense Not primer (980451):

5′-AGCGGCCGCTCAGTTGTTTTCCAAGTC-3′ (SEQ ID NO:26)

The amplification reaction (50 μl) was composed of the following components: 1×Pwo buffer (Roche Molecular Biochemicals, Indianapolis, Ind.), 0.2 mM dNTPs, 1.0 PM of each primer, 5 units of Pwo DNA polymerase, and approximately 60 ng of heat-denatured clone #13. The reactions were incubated in a Perkin-Elmer Model 480 Thermal Cycler programmed as follows: 22 cycles each at 94° C. for 45 seconds; 55° C. (52° C. for first two cycles) for 45 seconds; 72° C. for 90 seconds, and a final extension at 72° C. for 7 minutes.

The products were fractionated on a 0.8% agarose gel using TAE buffer, and the predominant band at 1.3 kb was excised and purified using the QIAquick Gel Extraction Kit. The products were cloned into pCR° 2.1-TOPO (Invitrogen, Carlsbad, Calif.) after addition of 3′ A-overhangs according to the manufacturer's suggested protocol.

The 1.3 kb insert from one randomly selected clone was removed by sequential digestion with SwaI and PacI (TAKA promoter construct) or NotI (niaA promoter construct), gel purified, and ligated into similarly digested pBANe13, pBANe44, or pPaHa3B. The ligation mixtures were transformed into E. coli DH5a, and clones were screened for the correct inserts by digestion with SwaI and PacI or SwaI and NotI. Miniprep DNA was sequenced from the ends of both inserts and shown to contain the full rdhA coding sequence. The constructs were designated pBANe13rad51, pSMO143, and pPaHa3Brad51.

The 1.96 kb coding region of rdhB was amplified essentially as described above using pZL1 clone #6 and the following primers:

Sense SwaI primer (980924):

5′-ATTTAAATGATGCCCMCACGACAGACA-3′ (SEQ ID NO:27)

Antisense PacI primer (980925):

5′-TTAATTMCTATTGCGGATGTTGTTGCT-3′ (SEQ ID NO:28)

Antisense NotI primer (980826):

5′-GCGGCCGCCTATTGCGGATGTTGTTGC-3′ (SEQ ID NO:29)

The annealing temperature for the PCR was 60° C. (58° C. for first two cycles). The DNA was subcloned into pCR-Blunt (Invitrogen, Carlsbad, Calif.), and miniprep DNA from clones containing the correct inserts was cloned into pBANe13, pBANe44, pRaMB33, or pPaHa3B as described above. The resulting constructs were named pBANe13rad52, pSMO145, pSMO155 and pPaHa3Brad52, respectively.

### Example 12

### Construction of Aspergillus oryzae PaHa29

Aspergillus oryzae hemA 5′-deletion strain SE29-70 (Elrod et al, 2000, Current Genetics 38:291-298) was cultured on PDA plates containing 5-aminolevulinic acid and uridine to allow for loss of the pyrG gene. Spores from this plate were then plated on minimal plates containing fluoroorotic acid (FOA), uridine, and 5-aminolevulinic acid. Eight FOA-resistant colonies were spore purified on minimal plates containing 5-aminolevulinic acid and uridine. One of the FOA-resistant colonies was verified as having a pyrG deletion phenotype by lack of growth on minimal medium containing 5-aminolevulinic acid and by recovery of prototrophy after transformation of protoplasts (prepared as in Example 13) with an autonomously-replicating plasmid carrying the pyrG gene (pHB217). This strain was designated Aspergillus oryzae PaHa29.

### Example 13

### Construction of Aspergillus oryzae HowB423 and HowB425

Protoplasts of Aspergillus oryzae HowB101 were transformed with pSMO143 or pSMO145 and plated on Basta transformation plates.

Protoplasts of Aspergillus oryzae strain HowB101 were prepared according to the method of Christensen et al., 1988, Bio/Technology 6: 1419-1422. The transformation was conducted with protoplasts at a concentration of ca. 2×10⁷ protoplasts per ml. One hundred μl of protoplasts were placed on ice for 5 minutes with ca. 2 μg of the pSMO143 or pSMO145; 250 μl of 60% polyethylene glycol 4000, 10 mM Tris-HCl, pH 7.5, 10 mM CaCl₂ was added, and the protoplasts were incubated at 37° C. for 30 minutes. Three mis of STC (1.2 M sorbitol, 10 mM Tris-HCl, pH 7.5, and 10 mM CaCl₂) was added. The solution was mixed gently and poured onto 150 mm Basta transformation plates (per liter: 0.52 g of KCl, 0.52 g of MgSO₄. 7H₂O, 1.52 g of KH₂PO₄, 1 ml of trace metals described below, 342.3 g of sucrose, 25 g of Noble agar, 10 ml of 1 M urea, 10 ml of 5 mg/ml Basta). The trace metals solution (1000×) was comprised of 22 g of ZnSO₄.7H₂O, 11 g of H₃BO₃, 5 g of MnCl₂.4H₂O, 5 g of FeSO₄.7H₂O, 1.6 g of CoCl₂.5H₂O, 1.6 g of (NH₄)₆Mo₇O₂₄, and 50 g of Na₄EDTA per liter. Plates were incubated 5-7 days at 34° C. until colonies appeared. Putative transformants were spore purified twice on the same medium.

### Example 14

### Construction of hemA 3′-Deletion

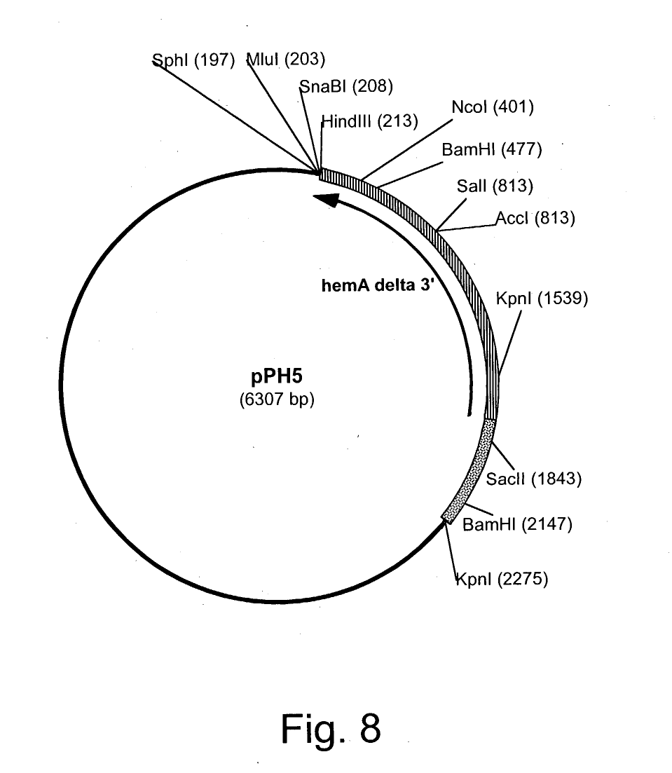

Plasmid pSE17 (WO 97/47746) was digested with HindIII to remove a portion of the hemA coding sequence and all of the 3′ flanking sequence to produce a 6.3 kb fragment. The 6.3 kb fragment was run on a 0.8% agarose gel in TAE buffer, excised, and purifed using a QIAEX II Gel Extraction Kit (QIAGEN, Chatsworth, Calif.) according to the manufacturer's instructions. The fragment was recircularized by ligation and transformed into E. coli XL1-Blue cells to yield plasmid pPH5 (FIG. 8).

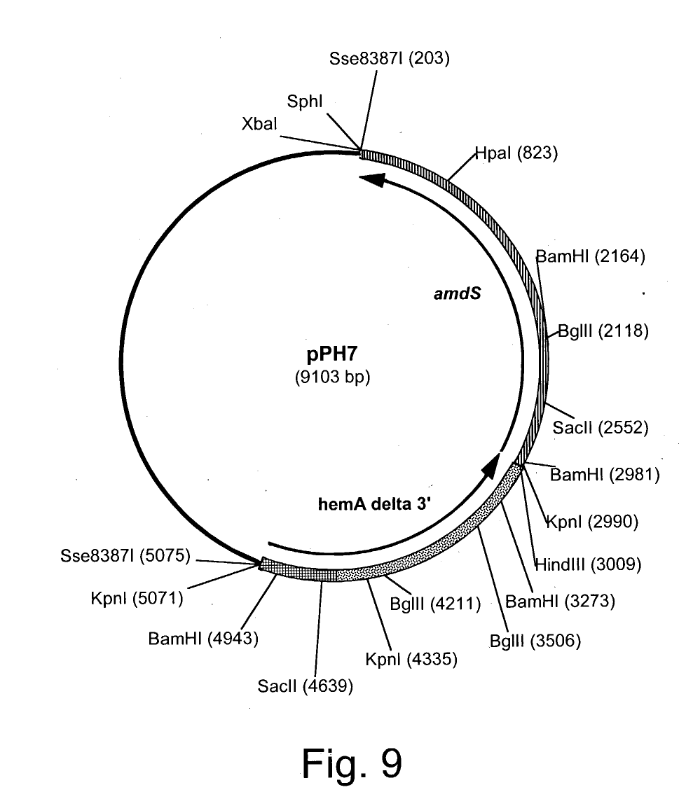

The amds gene from Emericella nidulans was isolated from pToC202 by digestion with EcoRI, Klenow fill-in, digestion with SphI, and gel purification as above. The amds gene fragment was ligated into pPH5 digested with SphI and SnaBI and similarly gel purified. The ligation mixture was transformed into E. coli XL1-Blue cells and plasmid DNA was prepared from twenty-four transformants. One plasmid DNA preparation showing the correct size fragments upon digestion with SacI, KpnI, or BamHI was designated pPH7 (FIG. 9).

### Example 15

### Construction of hemAA Strains Over expressing rdhA or rdhB

Protoplasts of Aspergillus oryzae PaHa29 were prepared as described in Example 13 and transformed with several μg of supercoiled pBANe13rad51, pBANe13rad52, 35 pPaHa3Brad51, or pPaHa3Brad52, and plated on minimal medium containing 30 μg/ml 5-aminolevulinic acid. Individual transformants were spore purified on MMGAS (per liter: 0.5 g of NaCl, 0.5 g of MgSO₄.7H₂O, 2.0 g of KH₂PO₄, 1.2 g of K₂HPO_{4,} 1 ml of trace metals described below, 218 g of sorbitol, 20 g of Noble agar, 3.7 g of NH₄Cl, 0.1 ml of 1.0 M CaCl₂, and 10 ml of glycerol) plus 5-aminolevulinic acid (pBANe13 transformants) or MMASM (per liter: 0.5 g of NaCl, 0.5 g of MgSO₄.7H₂O, 2.0 g of KH₂PO₄, 1.2 g of K₂HPO_{4,}1 ml of trace metals described below, 20 g of sucrose, 20 g of Noble agar, 3.7 g of NH₄Cl, and 0.1 ml of 1.0 M CaCl₂) plus 5-aminolevulinic acid (pPaHa3B transformants). The trace metals solution (1000×) was comprised of 10 g of ZnSO₄.7H₂O, 0.4 g of CuSO₄. 5H₂O, 0.04 g of Na₂B₄O₇.10H₂O, 0.7 g of MnSO₄.H₂O, 1.2 g of FeSO₄. 7H₂O, 1.6 g of CoCl₂.5H₂O, and 0.8 g of Na₂MoO₂.2H₂O per liter. Respective transformants from the indicated plasmids were designated PaHa3O, PaHa31, PaHa32, and PaHa33. Multiple transformants of each were generated and are designated by appending a number, e.g., PaHa31-2.

### Example 16

### Effect of rdhA or rdhB Overexpression on Interplasmid Recombination

Aspergillus oryzae grows very poorly using acetamide as the sole nitrogen source. Growth can be greatly enhanced by introduction of one or more copies of the amds gene from Emericella nidulans. This characteristic was used to monitor inter-plasmid recombination by co-transforming Aspergillus oryzae protoplasts with two autonomously-replicating plasmids, one carrying a deletion in the 5′ region of amds (pSMO145), and the other carrying a deletion in the 3′ region (pSMO146). Vigorous growth of transformants on acetamide can only be achieved following homologous recombination between the different plasmids to reconstitute at least one complete amds gene. Both plasmids also carry the pyrG gene in order to assess relative transformation efficiency.

The frequency of recombination in parental (Aspergillus oryzae HowB101) and rdhA (Aspergillus oryzae HowB443) or rdhB (Aspergillus oryzae HowB445) over-expression strains was assessed by co-transforming with both plasmids and plating on minimal medium with either nitrate or acetamide as the sole nitrogen sources (Table 1). The sucrose in these plates partially induces the TAKA promoter. Protoplasts of the indicated strains were prepared as described in Example 13 and co-transformed with 1.5 μg each of pSMO145 and pSMO146. A portion of the protoplasts was plated on minimal medium with either nitrate or acetamide as the sole nitrogen source, and the number of colonies was counted after six days of incubation at 37° C. Minimal nitrate plates contained, per liter, 6 g NaNO₃, 0.52 g KCl, 6.08 g KH₂PO₄, 0.5 g MgSO₄. 7H₂O, 342.3 g sucrose, 10 g glucose, 0.004 g biotin, 20 g noble agar, and 1 ml of the trace metals described in Example 15. The medium was adjusted to pH 6.5 with NaOH. Minimal acetamide plates (COVE) contained, per liter, 10 mM acetamide, 15 mM CsCl, 0.52 g KCl, 1.52 g KH₂PO₄, 0.52 g MgSO₄.7H₂O, 342.3 g sucrose, 25 g noble agar, and 1 ml of trace metals. Transformation with either plasmid alone yielded no transformants on acetamide. Overall transformation efficiency of the over-expressing strains was somewhat reduced compared to the parental strain, however, inter-plasmid recombination frequencies were elevated by 14 and 26-fold in the rdhA and rdhB over-expression strains, respectively. In Aspergillus oryzae HowB445, plasmids in almost half of the total transformants presumably underwent at least one homologous recombination event that reconstituted a functional amds gene.

TABLE 1
Stimulation of interplasmid recombination in rdhA or rdhB
overexpressing strains.

| | HowB101 | HowB443 | HowB445 |
|---|---|---|---|
| Transformants per ng, | 3.43 | 1.83 | 1.33 |
| nitrate (pyrG selection) | | | |
| Transformants per ng, acetamide | 0.06 | 0.46 | 0.61 |
| (amds and pyrG selection) | | | |
| Recombination frequency | 0.016 | 0.251 | 0.456 |
| Fold stimulation | 1.0 | 14.4 | 26.1 |

### Example 17

### Effect of rdhA or rdhB Overexpression on Interchromosomal Recombination

The hemA gene of Aspergillus oryzae codes for 5-aminolevulinate synthase, the first enzyme in heme biosynthesis. Mutants lacking this enzyme are unable to grow unless the medium is supplemented with 5-aminolevulinic acid. The native hemA gene in the rdhB overexpressing Aspergillus oryzae strain PaHa31-2 has been replaced by hemA carrying a 445-bp deletion in the 5′ region of the coding sequence according to the procedure described in U.S. Pat. No. 6,100,057, and thus this strain will not grow on minimal medium. Protoplasts of Aspergillus oryzae PaHa31-2 were transformed with 5 μg of plasmid pPH7 (Example 14) using the protocol described in Example 13. This plasmid carries the hemA gene with a deletion of all of the 3′-untranslated region and the last 382 bp of the coding region. The plasmid also contains the E. nidulans amds gene, and transformants were therefore initially selected on COVE plates (Example 16) containing 20 μg/ml of 5-aminolevulinic acid. One specific transformant that grew on COVE but still required 5-aminolevulinic acid for growth was spore purified twice and designated Aspergillus oryzae PaHa31-2.2.

Spores from transformant Aspergillus oryzae PaHa311-2.2 were plated on MMGU medium (MMGAS (Example 15) without sorbitol and with 10 mM urea in place of NH₄Cl) containing increasing concentrations of maltose in order to induce expression of rdhB in a controlled fashion. Growth on this medium can only occur if homologous recombination occurs between the single-copy chromosomal hemAΔ5′-gene and the chromosomally-integrated plasmid carrying the hemAΔ3′ gene.

The results demonstrated that induction of rdhB expression greatly increased the frequency of homologous recombination. Concentrations of maltose as low as 0.02% had an obvious stimulatory effect. Most of the colonies were very slow to first appear and also grew very slowly, even when transferred to new plates not containing maltose. However, these colonies grew fairly normally when the medium was supplemented with 5-aminolevulinic acid, indicating that the complementation for hemA deficiency was only partial. Most likely this resulted from a gene conversion event that restored the coding region of hemA in one of the hemA3′ gene copies, but failed to restore the 3′-untranslated region. This could result in relatively low-level expression and incomplete complementation.

The low concentrations of maltose required to achieve marked stimulation of hemA⁺ colony formation suggested that relatively mild induction of rdhB transcription was sufficient to maximally promote homologous recombination. Also, transcription from the TAKA promoter was not completely suppressed in glycerol, and thus the background levels of recombination seen on glycerol may at least partially reflect this lack of complete suppression. To overcome this, strains were created wherein rdhA (PaHa32) or rdhB (PaHa33) was expressed under control of the weaker niaA promoter. The 3′-deleted copy of hemA carried on plasmid pPH7 was introduced into these strains in a manner identical to that described above for creation of PaHa31-2.2. The specific transformants selected for testing were designated Aspergillus oryzae PaHa324.6 and PaHa33-5.1.

Approximately 2×10⁷ spores of PaHa32-4.6 or PaHa33-5.1 were plated on either MMASM (Example 15) or MMNSM (MMASM with 10 mM NaNO₃ in place of NH₄Cl). The former medium keeps the niaA promoter turned off and the latter medium induces the niaA promoter and hence stimulates transcription of the rdhA or rdhB gene. The appearance of colonies was monitored for 7 days. The results demonstrated that interchromosomal recombination is stimulated by an elevation in transcription of either rdhA or rdhB.

### Example 18

### PCR Amplification of a Portion of the Aspergillus oryzae rdhd Gene

A portion of the Aspergillus oryzae rdhD (rad54 homolog D) gene was amplified by nested degenerate PCR. The amplification employed primers 980057, 980058, 980059 and 980060 shown below.

Primer 980057:

5′-GAYCCIGAYTGGAAYCCNG-3′ (SEQ ID NO:30)

Primer 980058:

5′-TTYTTYTGICCRTCNCKCCA-3′ (SEQ ID NO:31)

Primer 980059:

5′-MYTAYACICARACNYTNGA-3′ (SEQ ID NO:32)

Primer 980060:

5′-ATITTYTCYTCDATNGTNC-3′ (SEQ ID NO:33)

The first amplification reaction (30 μl) was prepared using Aspergillus oryzae HB101 genomic DNA as template with the following components: PCR buffer II (Perkin Elmer), 0.20 mM dNTPs, 0.4 μg of Aspergillus oryzae HowB101 genomic DNA, 5.0 μM primer 980059, 5.0 μM primer 980060, and 3.0 units of Taq DNA polymerase. Before amplification, the template DNA was denatured in a boiling water bath for 5 minutes and quick-cooled on ice. The reaction was initiated by adding Taq DNA polymerase to the other reaction components at 72° C. The reactions were incubated in a Stratagene Robocycler programmed as follows: 35 cycles each for 45 seconds at 94° C., 45 seconds at 39,41, or 43° C., and 60 seconds at 72° C. (7 minutes final extension). Reaction products were pooled, precipitated with 2 volumes of ethanol, dried, and dissolved in 10 μl of TE. The second amplification reaction (30 pi) was prepared using the product of the first amplification as template with the following components: PCR buffer II (Perkin Elmer),0.20 mM dNTPs, 0.2 μl of template DNA, 5.0 μM primer 980057,5.0 μM primer 980058, and 3.0 units of Taq DNA polymerase. Before amplification, the template DNA was denatured in a boiling water bath for 5 minutes and quick-cooled on ice. The reaction was initiated by adding Taq DNA polymerase to the other reaction components at 72° C. The reactions were incubated in a Stratagene Robocycler programmed as follows: 35 cycles each for 45 seconds at 94° C., 45 seconds at 46, 48, 50, or 52° C., and 60 seconds at 72° C. (7 minutes final extension).

A portion of the reaction products was fractionated on a 3% agarose gel, and bands at about 70 bp were excised and purified using QIAquick with a final elution volume of 30 μl. Approximately 2 μl of this product was reamplified under the same PCR conditions and fractionated and purified in the same manner. The ca. 70 bp fragment was cloned using the TOPO TA cloning kit according to the manufacturer's instructions and the DNA sequence was determined using T7 promoter primer. DNA sequence analysis of the 68 bp gene segment showed that the amplified gene encoded a portion of the Aspergillus oryzae rdhD gene. The sequence from this clone was used to design a non-degenerate primer to be used for amplification of a larger region of the rdhD gene. The employed primer is shown below.

Primer 980866:

5′-MTGCTTGTTGATCAGCAG-3′ (SEQ ID NO:34)

The amplification reaction (120 μl) was prepared using Aspergillus oryzae HB425 genomic DNA as template with the following components: PCR buffer II (Perkin Elmer), 0.25 mM dNTPs, 2.0 μg template DNA, 4.2 μM primer 980059, 0.4 μM primer 980866, and 5.0 units of Taq DNA polymerase. Before amplification, the template DNA was denatured in a boiling water bath for 5 minutes and quick-cooled on ice. The reaction was initiated by adding Taq DNA polymerase to the other reaction components at 72° C. The reactions were incubated in a Stratagene Robocycler programmed as follows: 30 cycles each for 45 seconds at 94° C., 45 seconds at 39, 41, 43, or 45° C., and 60 seconds at 72° C. (7 minutes final extension). The ca. 250 bp product was fractionated on an agarose gel, excised, and purified using the QIAquick system. Three μl of the purified fragment was reamplified under the same PCR conditions for 25 cycles at an annealing temperature of 40° C., and the product was gel purified in the same manner. Direct sequencing of the PCR product using primer 980866 demonstrated that the gene fragment encoded a portion of the rdhD gene.

### Example 19

### Isolation of Partial-Length Aspergillus oryzae rdhD Genomic Clones

Genomic libraries were prepared and plated as in Example 3. The PCR product of 232 bp described in Example 18 was radioactively labeled using the Stratagene Prime-It II kit according to the manufacturer's protocol with the exception that the random primers were replaced by 0.6 μM of primer 866. The labeled product was used to probe approximately 100,000 pfu of an Aspergillus oryzae genomic library constructed from Aspergillus oryzae strain HowB430 in the vector λZipLox using the same procedures described in Example 3.

Eleven hybridizing plaques were obtained, and four of these were purified, excised as pZL1 clones, and characterized as described in Example 3.

### Example 20

### Characterization of the Aspergillus oryzae Genomic Clone Encoding RDHD

DNA sequencing of each clone was performed with an Applied Biosystems Prism 377 DNA Sequencer using the BigDye Terminator Cycle Sequencing Ready Reaction kit according to the manufacturer's instructions. Oligonucleotide sequencing primers were designed to complementary sequences in the pZL1 plasmid vector and were synthesized by Operon Technologies Inc., Alameda, Calif. Contig sequences were generated using a transposon insertion strategy (Primer Island Transposition Kit, Perkin-Elmer/Applied Biosystems, Inc., Foster City, Calif.).

A 5514 bp genomic fragment was sequenced to an average redundancy of 6.0, and includes sequences from all of the genomic clones. No single clone contained the entire gene, but overlapping pZL1 clones #10 and #17 together encompassed the entire gene. The nucleotide sequence and deduced amino acid sequence are shown in FIG. 2. Sequence analysis of the cloned insert revealed a coding sequence of 2645 bp (excluding the stop codon) encoding a protein of 811 amino acids. Clone 10 contained nucleotides 390-2906 of SEQ ID NO:5 encoding amino acids 59-811 of SEQ ID NO:6, while clone 17 contained nucleotides 161-1749 of SEQ ID NO:5 encoding amino acids 1-459 of SEQ ID NO:6. The coding sequence is punctuated by four introns of 54 bp, 63 bp, 49, and 46 bp. The G+C content of the coding sequence (including introns) is 47.3%. The predicted RDHD polypeptide has a molecular mass of 99.2 kDa and an isoelectric point of 8.90. Using the Signal P software program (Nielsen et al., 1997, Protein Engineering 10:1-6), no signal peptide was predicted (Y<0.037).

A comparative alignment of the Aspergillus oryzae RDHD protein sequence with other sequences using the Clustal W algorithm in the Megalign program of DNASTAR, showed that the deduced amino acid sequence of the Aspergillus oryzae RDHD protein shares 74% identity to the deduced amino acid sequence of the MUS-25 protein of Neurospora crassa (accession number Q9P978).

Clones 10 and 17 were deposited as E. coli pZL1rdhD17 (NRRL B-30505) and E. coli pZL1rdhD10 (NRRL B-30506) on Jul. 27, 2001, with the Agricultural Research Service Patent Culture Collection, Northern Regional Research Center, 1815 University Street, Peoria, Ill.

### Example 21

### Construction of pHB241 and pHB242

pToC202 was digested with HindIII and then shrimp alkaline phosphatase (Roche, Indianapolis, Ind.) was added and incubated according to the manufacturer's instructions. The 5.4 kb fragment was agarose gel purified using Qiex II (QIAGEN, Chatsworth, Calif.

pHB217 (Example 10) was digested with HindIII endonuclease. The 5.8 kb fragment containing the Aspergillus oryzae AMAI region was gel-isolated using Qiex II.

The 5.4 kb and 5.8 kb fragments were ligated for two hours and used to transform One Shot competent E. coli (Invitrogen, Carlsbad, Calif.) according to the manufacturer's instructions. The plasmid was designated pHB241.

Plasmid pHB241 was digested with both NheI and BstEII and the ends were made blunt using the Klenow fragment of DNA Polymerase I. The plasmid was closed by ligation and designated pHB242.

### Example 22

### Construction of pPAHA1 Step 1

Plasmid pBANe6 (U.S. Pat. No. 5,958,727) was digested with BamHI and BseRI and the ends were filled in with T4 DNA polymerase. A 6.75 kb fragment was gel-purified and isolated using the Qiaquick system. The fragment was ligated and transformed into E. coli Sure Cells (Strategene, La Jolla, Calif.) following manufacturer's instructions. The resulting plasmid was named pPAHA1 Step1, which contains a 222 bp deletion of the amds gene.

### Example 23

### Gap Repair in Aspergillus oryzae

The following experiments were performed to determine whether Aspergillus oryzae functions as a host for gap repair and DNA shuffling.

Two amds deletion fragments were derived from HpaI/EcoRI digestion of pToc202 and NsiI digestion of pPaHa1 to yield 813 bp of 5′ and 604 bp of 3′ overlap with the gapped plasmid pHB241, respectively, and resolved using 1% agarose gels. The fragments were cut from the gel and the DNA was purified using the Qiaex II Gel-Extraction Kit as recommended by manufacturer (Qiagen, Chatsworth, Calif.). These two fragments were co-transformed with the NheI/BstEII digested plasmid pHB241 or pHB242, a re-ligated gapped pHB241 linearized with BssHII (Table 1) into Aspergillus oryzae host cells described in Table 1 using the same protoplast techniques described in Example 13. Transformants were selected on Cove+uridine for growth on acetamide (Example 16).

The results are shown below in Table 1.

TABLE 1
Co-transmformants of amds deletion fragments and linear gapped
pHB241 and pHB242

| | | |
|---|---|---|
| Strain | DNA | Transformants |

Experiment 1

| | | |
|---|---|---|
| A. oryzae HowB101 | pHB241 circular (100 ng) | 500 |
| | PHB242 linear (300 ng) | 0 |
| PToC202 EcoRI/HpaI | 0 | |
| (E/H) (400 ng) + | | |
| | pPaHa1 NsiI (N) (400 ng) | |
| | pHB242 linear (300 ng) + | 55 |
| | pToC202 E/H, pToC202 | |
| | N (400 ng each) | |
| A. aryzae HowB445 | pHB241 circular (100 ng) | 75 |
| | pHB242 linear (300 ng) + | 25 |
| | pToC202 E/H, pToC202 | |
| | N (400 ng each) | |

Experiment 2

| | | |
|---|---|---|
| A. oryzae HowB101 | pHB241 circular (10 ng) | 77 |
| | pHB241 linear (150 ng) | 5 |
| | pToC202 E/H (200 ng) + | 0 |
| | pPaHa1 NsiI (200 ng) | |
| | pHB241 linear (150 ng) + | 89 |
| | pToC202 E/H, pToC202 | |
| | N (200 ng each) | |
| Aspergillus oryzae | | |
| HowB445 | | |
| | pHB241 circular (10 ng) | 5 |
| | pHB241 linear (150 ng) | 0 |
| | pHB241 linear (150 ng) + | 45 |
| | pToC202 E/H, pToC202 | |
| | N (200 ng each) | |

The results of experiments 1 and 2 demonstrate that recombination occurred between the two deletion bearing fragments and the gapped plasmid, restoring a functional amds gene. Because of the different transformation frequencies exhibited between the wild type and rdhB strains, recombination efficiencies were calculated as the percentage of amds recombinants per ng normalized to the strains transformation efficiency. In experiment 1, a three-fold increase in recombination was achieved. In experiment 2, recombination in the rdhB over-expressing strain is increased approximately seven-fold. In all experiments, no amds recombinants were ever observed in transformations using the two amds deletion fragments alone. These results confirm that, as in yeast, DNA shuffling is a process that occurs in Aspergillus oryzae.

The total number of transformants was consistently higher in Aspergillus oryzae HowB101. This is most likely due to the higher transformation frequency of this strain. Increasing the amount of either the gapped plasmid or linear DNA's resulted in greater numbers of transformants. In several experiments, up to 500 recombinants were obtained. These high numbers suggest that sufficient yields of DNA shuffled recombinants could be obtained directly in Aspergillus oryzae for screening as opposed to heterologous systems such as yeast.

Also apparent is the higher proportion of amds recombinants obtained in the rdhB over-expression Aspergillus oryzae strain HowB 445. With transformation frequencies approximately 10-fold lower, the rdhB over-expression strain generated amds recombinants equal or greater than that obtained in a wildtype strain. From this it can be concluded that increased expression of recombination-associated genes stimulate homologous recombination in Aspergillus oryzae.

### Example 24

### Construction of Plasmid pENi2229

Plasmid pENi2229 was constructed to incorporate additional restriction sites using several plasmids as described below. The final pENi2229 plasmid contains the AMA1 sequence for autonomous replication in Aspergillus species, a pyrG selectable marker for selection in filamentous fungi, a strong TAKA-npi promoter for the expression of proteins, a number of useful restriction sites downstream of the promoter, a termination sequence, an E. coli ori sequence for replication in bacteria, and a beta-lactamase expression cassette for selection in bacteria.

Using pENI2151 as template and PWO polymerase (as recommended by the manufacture), a PCR-reaction was made using primer 2120201J1 and 1288-taka.

1298-TAKA:

gcaagcgcgcgcaatacatggtgttttgatcat (SEQ ID NO:35)

210201J:

GCCTCTAGATCTCCCGGGCGCGCCGGCACATGTACCAGGTCTTAAGCTCGAGCTCGGTCACCGGTGGCC (SEQ ID NO:36)

The PCR fragment (650 bp) and pEN12207 were digested with restriction endonucleases BssHII and BgIII. The vector and the PCR fragment were purified from a 1% agarose gel using Qiagen spin columns (Qiagen, Valencia, Calif.) following the manufacturer's instructions.

The PCR fragment and the vector were ligated, and transformed into the E. coli strain DH10B. Plasmid from one of the transformants was isolated (Qiagen, Valencia, Calif.) following the manufacturer's instructions, verified by DNA sequencing, and named pEN12229.

Plasmid pENI2151: Plasmids pENI1902 and pENI1861 were both digested with restriction endonuclease HindIII, and.pENI1902 was treated with phosphatase. Both a 2408 bp fragment from pENI1861 and digested vector pENI1902 were purified from 1% gel using Qiagen spin columns (Qiagen, Valencia, Calif.) following the manufacturer's instructions.

The fragment and the vector were ligated, and transformed into the E. coli strain DH 10B. Plasmid from one of the transformants was isolated and named pENI2151.

Plasmid pENI2207: Plasmids pENI2151 and pENI2155 were digested with restriction endonucleases StuI and SphI. Both the 2004 bp fragment from pENI2155 and digested vector PENI2151 were purified from 1% gel using Qiagen spin columns (Qiagen, Valencia, Calif.) following the manufacturer's instructions. The fragment and the vector were ligated, and transformed into the E. coli strain DH10B. Plasmid from one of the transformants was isolated and named pEN12207.

Plasmid pENI1902 was made in order to have a promoter that works in both E. coli and Aspergillus. This was done by unique site elimination using the “Chameleon double stranded site-directed mutagenesis kit” as recommended by Stratagene®.

Plasmid pENI1861 was used as template and the following primers with 5′ phosphorylation were used as selection primers: 177996, 135640, and 135638. The 080399J19 primer with 5′ phosphorylation was used as mutagenic primer to introduce a −35 and −10 promoter consensus sequence (from E. coli) in the Aspergillus expression promoter. Introduction of the mutations was verified by sequencing.

177996: gaatgacttg gttgacgcgt caccagtcac (SEQ ID NO:37)

135640: cttattagta ggttggtact tcgag (SEQ ID NO:38)

135638: gtccccagag tagtgtcact atgtcgaggc agttaag (SEQ ID NO:39)

080399J19: gtatgtccct tgacaatgcg atgtatcaca tgatataatt actagcaagg gaagccgtgcttgg (SEQ ID NO:40)

Plasmid pENI1861 was made in order to have the state of the art Aspergillus promoter in the expression plasmid, as well as a number of unique restriction sites for cloning. A PCR fragment (Approx. 620 bp) was made using plasmid pMT2188 (the construction of pMT2188 is described below) as template and the following primers:

051199J1: cctctagatctcgagctcggtcaccggtggcctccgcggccgctggatccccagttgtg (SEQ ID NO:41)

1298TAKA: gcaagcgcgcgcaatacatggtgttttgatcat (SEQ ID NO:42)

The fragment was cut with BssHII and BgIII, and cloned into pEN11849 which was also cut with BssHII and BgIII. The cloning was verified by sequencing.

Plasmid pMT2188 was based on the Aspergillus expression plasmid pCaHj 483 (described in WO 98/00529), which consists of an expression cassette based on the Aspergillus niger neutral amylase II promoter fused to the Aspergillus nidulans triose phosphate isomerase non translated leader sequence (Pna2/tpi) and the Aspergillus niger amyloglycosidase terminater (Tamg). Also present on the pCaHj483 is the Aspergillus selective marker amds from A. nidulans enabling growth on acetamide as sole nitrogen source. These elements are cloned into the E. coli vector pUC19 (New England Biolabs). The ampicillin resistance marker enabling selection in E coli of pUC19 was replaced with the U RA3 marker of Saccharomyces cerevisiae that can complement a pyrF mutation in E. coli, the replacement was done in the following way:

The pUC19 origin of replication was PCR amplified from pCaHj483 with the primers:

142779: 5′ ttgaaftgaaaatagaftgatttaaaacttc-3′ (SEQ ID NO:43)

142780: 5′ ttgcatgcgtaatcatggtcatagc-3′ (SEQ ID NO:44)

Primer 142780 introduces a BbuI site in the PCR fragment. The Expand TM PCR system (Roche Molecular Biochemicals, Basel, Switzerland) was used for the amplification following the manufacturers instructions for this and the subsequent PCR amplifications.

The URA3 gene was amplified from the general S. cerevisiae cloning vector pYES2 (Invitrogen, Carlsbad, Calif., USA) using the primers:

140288: 5′ ttgaattcatgggtaataactgatat-3′ (SEQ ID NO:45)

142778: 5′ aaatcaatctattttcaattcaaftcatcatt-3′ (SEQ ID NO:46)

Primer 140288 introduces an EcoRI site in the PCR fragment. The two PCR fragments were fused by mixing them and amplifying using the primers 142780 and 140288 in the splicing by overlap method (Horton et al., 1989, Gene 77: 61-68).

The resulting fragment was digested with EcoRI and BbuI and ligated to the largest fragment of pCaHj 483 digested with the same enzymes. The ligation mixture was used to transform the pyrF E. coli strain DB6507 (ATCC 35673) made competent by the method of Mandel and Higa (Mandel and Higa, 1970. J. Mol. Biol. 45:154). Transformants were selected on solid M9 medium (Sambrook et. al., 1989, Molecular cloning, a laboratory manual, 2nd edition, Cold Spring Harbor Laboratory Press) supplemented with 1 g/l casaminoacids, 500 microgram/l thiamine and 10 mg/l kanamycin.

A plasmid from a selected transformant was termed pCaHj527. The Pna2/tpi promoter present on pCaHj527 was subjected to site directed mutagenises by a simple PCR approach. Nucleotides 134-144 were altered from GTACTAAAACC to CCGTTAAATTT using the mutagenic primer 141223. Nucleotides 423-436 were altered from ATGCAATTTAAACT to CGGCAATTTAACGG using the mutagenic primer 141222. The resulting plasmid was designated pMT2188.

Primer 141223:

5′ ggatgctgttgactccggaaatttaacggtttggtcttgcatccc-3′ (SEQ ID NO:47)

Primer 141222:

5′-ggtattgtcctgcagacggcaatttaacggcttctgcgaatcgc-3′ (SEQ ID NO:48)

Plasmid pENI1849 was made in order to truncate the pyrG gene to the essential sequences for pyrG expression, in order to decrease the size of the plasmid, thus improving transformation frequency. A PCR fragment (approx. 1800 bp) was made using pEN11299 (described in WO 00/24883, Example 1) as template and the following primers;

270999J8: tctgtgaggcctatggatctcagaac (SEQ ID NO:49)

270999J9: gatgctgcatgcacaactgcacctcag (SEQ ID NO:50)

The PCR-fragment was digested with StuI and SphI, and cloned into pENI1298 (described in WO 00/24883, Example 1), and also digested with StuI and SphI; the cloning was verified by sequencing.

Plasmid pEN12155 comprises a bad kozak region upstream of the pyrG gene, and is constructed as follows:

Using plasmid pENI1861 as template, and PWO polymerase (conditions as recommended by manufacturer), two PCR-reactions were made using primer 141200J1 and 270999J9 in the one PCR-reaction and primers 141200J2 and 290999J8 in another PCR-reaction:

141200J1: 5′-atcggttttatgtcttccaagtcgcaattg-3′ (SEQ ID NO: 51)

141200J2: 5′-cttggaagacataaaaccgatggaggggtagcg-3′ (SEQ ID NO:52)

270999J8: 5′-tctgtgaggcctatggatctcagaac-3′ (SEQ ID NO:53)

270999J9: 5′-gatgctgcatgcacaactgcacctcag-3′ (SEQ ID NO:54)

The PCR fragments were purified from a 1% agarose gel using QIAGEN™ spin columns. A second PCR-reaction was run using the two fragments as template along with the primers 270999J8 and 270999J9. The PCR-fragment from this reaction was purified from a 1% agarose gel as described; the fragment and the vector pEN11849 (containing a lipase gene as expression reporter) were cut with the restriction enzymes StuI and SphI, the resulting fragments were purified from a 1% agarose gel as described previously.

The purified fragments were ligated and transformed into the E. coli strain DH10B. Plasmid DNA from one of the transformants was isolated and sequenced to confirm the introduction of a mutated Kozak region: ggttttatg (rather than the wildtype: gccaacatg). This plasmid was denoted: pEN12155.

### Example 25

### Construction of pCW013

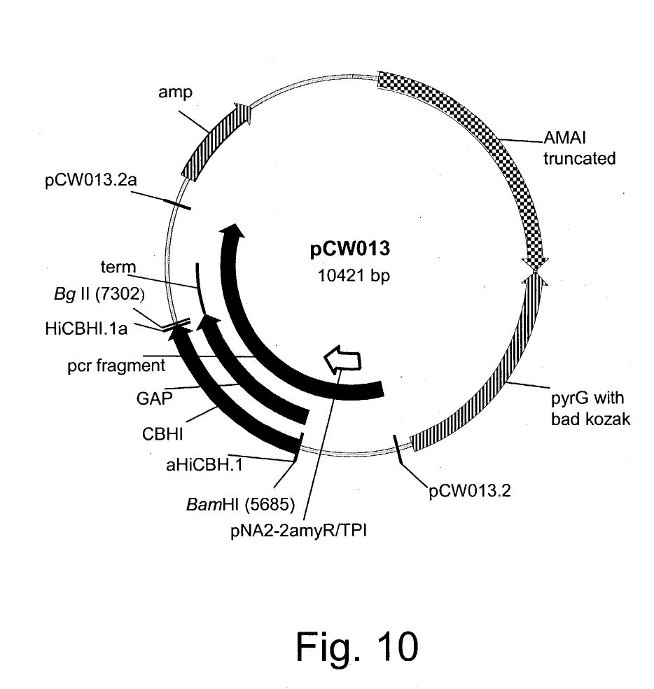

Plasmid pCW013 (FIG. 10) was constructed from pENi2229 to obtain expression of a Humicola insolens cellobiohydrolase (CBHI) in Aspergillus oryzae. The coding sequence for Humicola insolens CBHI was amplified by PCR from pHD459b, which was created as described by Dalboge and Heldt-Hansen, 1994, Mol. Gen. Gene 243: 253-260, utilizing the screening procedure for glucanase detection. The PCR fragment containing the full-length cbh1 gene was subcloned into pENi2229 as a BamHI/XmaI fragment. Construction of the pCWO13 plasmid was accomplished as described below.

PCR fragments were extended with a BamHI site on the 5′ end of CBHI and an XmaI site on the 3′ end using the following primers.

Primer 1: 5′-CGCGGATCCACCATGCGTACCGCCMGTTCGCC-3′ (SEQ ID NO:55)

Primer 2: 5′-GCCCCGGGTTACAGGCACTGAGAGTACCAG-3′ (SEQ ID NO:56)

The amplification reactions (50 μl) contained the following components: 0.3 μg of pHD459b1 unit of PWO polymerase, 1×PWO polymerase buffer, 0.2 mM dNTPs, 50 pmol of primer 1, and 50 pmol of primer 2. The reactions were incubated in a Eppendorf Mastercycler (Eppendorf, Westbury, N.Y.) programmed for 30 cycles each at 95° C. for 30 seconds, 55° C. for 30 seconds and 72° C. for 1 minute.

The reaction products were then resolved on a 0.8% agarose gel and a 1605 bp product band was excised from the gel and purified using Amicon's Ultrafree DA Centrifugal Unit (Millipore, Bedford, Mass.) according to manufacturer's instructions. The purified product was then ligated and transformed using pCR4 Blunt TOPO Vector Kit (Invitrogen, Carlsbad, Calif.) following the manufacturer's instructions. The transformation was plated on 2XYT/ampicillin agar medium and grown overnight at 37° C. The 2XYT/ampicillin agar medium was composed per liter of per liter 16 g of tryptone, 10 g of yeast extract, 5 g of sodium chloride, and 15 g of Bacto agar supplemented with 100 μg of ampicillin per liter.

White colonies were picked into 3 ml of 2XYT/ampicillin medium and grown overnight at 37° C. Plasmid DNA was isolated from the cultures using Qiagen Qiabot Miniprep Station (Qiagen, Valencia, Calif.) following the manufacturer's instructions. The plasmid DNA was analyzed by restriction mapping to identify clones positive for CBHI insertion using restriction endonucleases BamHI and XmaI. Once a clone was validated that there was successful insertion of the CBHI gene, the clone was sequenced for fidelity using BigDye Terminator Version 3 and analyzed using ABI 3700 DNA Analyzer (Foster City, Calif.) according the manufacturer's instructions.

The TOPO clone DNA containing confirmed CBHI sequence was digested with BamHI and XmaI and the reaction product was then resolved on a 0.8% agarose gel and a 1605 bp product was excised and purified using Amicon's Ultrafree DA Centrifugal Unit (Amicon, Beverly, Mass.) following the manufacturer's instructions.

Plasmid pENi2229 was digested in the same manner with BamHI and XmaI to create compatible ends with CBHI. The digestion product was resolved on a 0.8% agarose gel and an 8810 bp product was excised and purified using Amicon's Ultrafree DA Centrifugal Unit according to the manufacturer's instructions. The BamHI/XmaI CBHI gene fragment was ligated into the BamHI/XmaI digested pENi2229 using Rapid DNA Ligation Kit (Roche, Indianapolis, Ind.) following the manufacturer's instructions. This ligation was then used to transform E. coli Sure Cells following the manufacturer's instructions. Colonies were selected, cultured, and plasmid was prepared as described above. The plasmid DNA was analyzed by restriction mapping to identify clones positive for CBHI insertion using BamHI and XmaI. The positive colonies were designated pCWO13.

### Example 26

### Generation of Gap Repair Fragments

To examine whether Aspergillus oryzae functions as a host for gap repair, the following experiments were performed. The DNA fragment containing the cbh1 gene was derived by PCR using plasmid pCW013 as the template and the following primers that anneal 900 bp upstream and 1063 bp downstream of the gap generated from BamHI/BgIII digestion of pCW013:

Primer 3: 5′-cgatctcgcagtcccgaftcgcc-3′ (SEQ ID NO:57)

Primer 4: 5′-tccgggagctgcatgtgtcagag-3′ (SEQ ID NO:58)

Amplification reactions (50 μl) were composed of 0.5 μg of pCWO13,50 pmol of primer 3, 50 pmol of primer 4, 0.2 mM dNTP's, 1×Taq DNA polymerase buffer, and 2.5 Units of Taq DNA polymerase. The reactions were incubated in an Eppendorf Mastercycler programmed for 20 cycles each 94° C. for 30 seconds, 55° C. for 30 seconds, and 72° C. for 3 minutes. The reaction products were purified using Qiaquick PCR Purification Kit (Qiagen, Valencia, Calif.) following the manufacturer's instructions.

Gapped pCW013 was prepared using BamHI and BgIII as follows: 37 μg of pCW013,50 units of BamHI, 50 units of BgIII (Roche, Indianapolis, Ind.), and 1×BufferA (Roche, Indianapolis, Ind.) were incubated for 3 hours at 37° C. The reaction product was then resolved on a 0.8% agarose gel where a 8816 bp product band was excised from the gel and purified using Amicon's Ultrafree DA Centrifugal Unit according to manufacturer's instructions.

### Example 27

### Preparation and Transformation of Aspergillus oryzae

Protoplasts of Aspergillus oryzae Jal250 (WO 99/61651, Example 9) were prepared similarly as described in Example 13. Frozen protoplasts of Aspergillus oryzae Jal250 were thawed on ice. Gapped pCWO13 and the cbh1 PCR fragment, at approximately a 1:3 molar ratio, respectively, were added to a 15 ml sterile polypropylene tube. One hundred μl of protoplasts were added and mixed gently. Two-hundred-fifty μl of PEG solution was then added to the DNA, mixed gently, and incubated at 37° C. for 20 minutes. Three ml of STC was then added and mixed gently. Aliquots of 300 μl were removed and added to 20 ml of 50° C. pyrG overlay (per liter: 0.5 g of NaCl, 0.5 g of MgSO₄.7H₂O, 2.0 g of KH₂PO₄, 1.2 g of K₂HPO_{4,}1 ml of trace metals described below, 20 g of sucrose, 20 g of Noble agar, 3.7 g of NH₄Cl, and 0.1 ml of 1.0 M CaCl₂; the trace metals solution (1000×) was composed per liter of 10 g of ZnSO₄.7H₂O, 0.4 g of CuSO₄.5H₂O, 0.04 g of Na₂B₄O₇.10H₂O, 0.7 g of MnSO₄.2H₂O, 1.2 g of FeSO₄.7H₂O, 1.6 g of CoCl₂.5H₂O, and 0.8 g of Na₂MoO₂.2H₂O) which was then poured on room temperature plates. These plates were allowed to solidify at room temperature and then incubated at 34° C. for 3 days. The results are shown in Table 2.

TABLE 2

| | |
|---|---|
| Gapped pCW013 | 0 colonies |
| Gapped pCW013 + PCR fragment | 72 colonies |

The results indicated that reconstitution of an autonomously replicating plasmid required the presence of both the gapped pCWO13 and the cbh1 PCR fragment.

Activity assays were performed to validate the fidelity of the repair. The transformants should contain an expression cassette encoding CBHI that was contained on the PCR fragment. These transformants were isolated and grown in 24 well plates containing 1 ml of ¼ strength MDU2BP medium containing maltose to induce the production of CBHI. The plates were incubated at 34° C. for 4 days. Positive controls were six transformants containing intact pCW013, which are positive for CBHI production. The negative controls were six transformants containing pENi2229, which is negative for CBHI. The controls were obtained following the protoplasting and tranformation procedure described above, substituting 1 μg of plasmid DNA for the gapped plasmid/PCR fragment DNA mix. At 4 days, samples of the culture broth were assayed for CBHI activity.

CBHI activity was determined as follows. Broth samples were diluted in assay buffer to a final concentration of 50 mM succinate, pH 5.0 and 0.01% Tween-20. The substrate phosphoric acid swollen cellulose (PASC) was added at 0.5% (v/v). Following a 20 hour incubation at room temperature, reducing sugars were measured using the p-hydroxybenzoicacid hydrazide (PHBAH) method according to Lever, 1972, Anal. Biochem. 24: 273-279). The final concentration of reagents was 1.5% PHBAH, 2% NaOH, and 5% potassium sodium tartrate tetrahydrate. The reactions were heated at 100° C. for 10 minutes, and sample absorbance measured at 405 nm. To determine reducing sugar release due to CBHI activity, control samples lacking PASC were subtracted from samples containing PASC. Final enzyme activity levels were compared to that obtained from culturing pCW13 (non-gap repaired) transformants. Relative absorbance values were indicative of enzyme activity, relating directly to the moles of reducing sugars released by CBHI degradation of PASC.

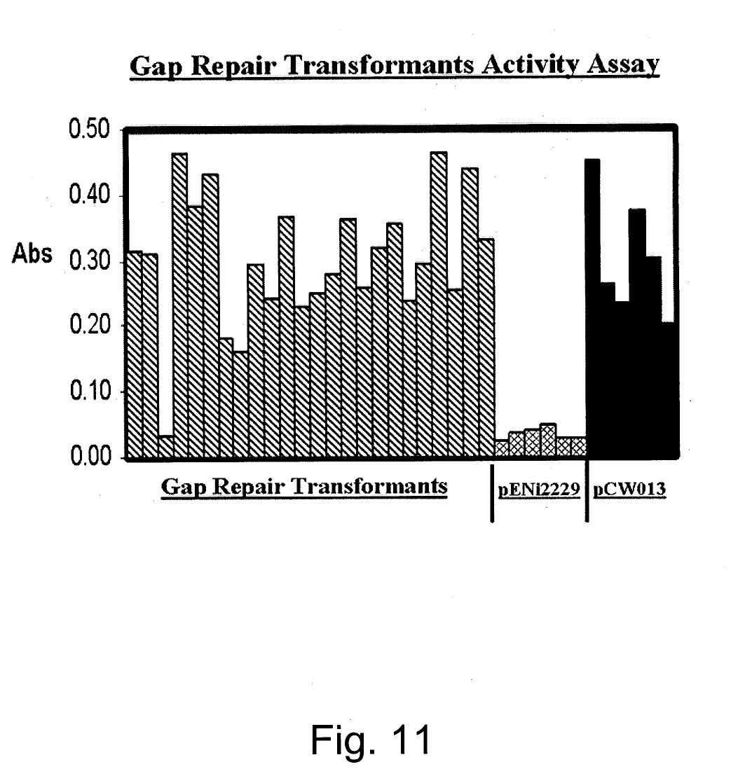

The assay results shown in FIG. 11 demonstrated that repair of the gap was complete and that gap repaired plasmids expressed CBHI at levels comparable to transformants harboring the circular plasmid pCW013.

### Deposit of Biological Material

The following biological material has been deposited under the terms of the Budapest Treaty with the Agricultural Research Service Patent Culture Collection, Northern Regional Research Center, 1815 University Street, Peoria, Ill., 61604, and given the following accession numbers:

| Deposit | | Accession Number | Date of Deposit |
|---|---|---|---|
| | E. coli pZL | NRRL B-30503 | Jul. 27, 2001 |
| | 1rdhA13 | | |
| | E. coli pZL | NRRL B-30504 | Jul. 27, 2001 |
| | 1rdhB6 | | |
| | E. coli pZL | NRRL B-30505 | Jul. 27, 2001 |
| | 1rdhD17 | | |
| | E. coli pZL | NRRL B-30506 | Jul. 27, 2001 |
| | 1rdhD10 | | |

The strains have been deposited under conditions that assure that access to the culture will be available during the pendency of this patent application to one determined by the Commissioner of Patents and Trademarks to be entitled thereto under 37 C.F.R. §1.14 and 35 U.S.C. §122. The deposits represent a substantially pure cultures of the deposited strains. The deposits are available as required by foreign patent laws in countries wherein counterparts of the subject application, or its progeny are filed. However, it should be understood that the availability of a deposit does not constitute a license to practice the subject invention in derogation of patent rights granted by governmental action.

The invention described and claimed herein is not to be limited in scope by the specific embodiments herein disclosed, since these embodiments are intended as illustrations of several aspects of the invention. Any equivalent embodiments are intended to be within the scope of this invention. Indeed, various modifications of the invention in addition to those shown and described herein will become apparent to those skilled in the art from the foregoing description. Such modifications are also intended to fall within the scope of the appended claims. In the case of conflict, the present disclosure including definitions will control.

Various references are cited herein, the disclosures of which are incorporated by reference in their entireties.

What is claimed is:

1. A method for preparing variants of a nucleotide sequence in a filamentous fungal host, comprising:
(a) introducing into a population of filamentous fungal host cells:
(i) one or more circular plasmids comprising a DNA sequence and a plasmid replicator mediating autonomous replication, wherein the one or more circularized plasmids are linearized by digestion of the DNA sequence and removal of a portion of the DNA sequence; and
(ii) a library of DNA fragments comprising one or more mutations of the DNA sequence, wherein the fragments comprise at least two regions, one or more regions which are homologous to the 5′ region or the 3′ region of the gap in the linearized DNA sequence and/or plasmid sequence and one or more second regions which are homologous to the 5′ region or the 3′ region of the DNA fragments of the library;
wherein the linearized plasmids and the DNA fragments recombine by in vivo recombination to produce a plurality of autonomously replicating plasmids comprising one or more variants of the DNA sequence;
(b) cultivating the population of recombinant filamentous fungal cells in a medium suitable for growth; and
(c) screening the population of recombinant filamentous fungal cells for variants of the DNA sequence contained on one or more autonomously replicating circularized plasmids.

2. The method of claim 1, wherein more than one cycle of steps (a) to (c) is performed.

3. The method of claim 1, wherein two or more linearized plasmids are recombined by in vivo recombination with two or more homologous DNA fragments in the same cycle.

4. The method of claim 1, wherein the ratio between the linearized plasmids and the homologous DNA fragments are in the range from 20:1 to 1:50 mol plasmid:mol fragments with specific concentrations in the range of 1 μM to 10 M of the DNA.

5. The method of claim 1, wherein at least 2 of the DNA fragments have partially overlapping regions.

6. The method of claim 1, wherein 2 to 50 of the DNA fragments have partially overlapping regions.

7. The method of claim 1, wherein 2 to 10 of the DNA fragments have partially overlapping regions.

8. The method of claim 1, wherein the overlapping regions of the DNA fragments are in the range from 30 to 5000 bp.

9. The method of claim 1, wherein the overlapping regions of the DNA fragments are in the range from 30 bp to 500 bp.

10. The method of claim 1, wherein the overlapping regions of the DNA fragments are in the range from 30 bp to 100 bp.

11. The method of claim 1, wherein at least one cycle of step (a) to (c) is backcrossing with the initially used DNA fragments.

12. The method of claim 1, wherein the DNA fragments are prepared under conditions suitable for high, medium or low mutagenesis.

13. The method of claim 1, wherein the DNA sequence is selected from the group consisting of (a) a gene that encodes a polypeptide or an RNA; (b) a disrupted gene; (c) a partially deleted gene; (d) a regulatory control sequence; (e) a recombinantly manipulated version of a gene native or foreign to the filamentous fungal host cell; (f) a transposon; (g) a ribozyme; or (h) a portion of (a), (b), (c), (d), (e), (f) or (g).

14. The method of claim 13, wherein the polypeptide is an antibody, hormone, enzyme, receptor, reporter, selectable marker, or a protein with biological activity.

15. The method of claim 14, wherein the enzyme is an oxidoreductase, transferase, hydrolase, lyase, isomerase, or ligase.

16. The method of claim 13, wherein the regulatory control sequence is selected from the group consisting of a promoter, signal sequence, leader, polyadenylation sequence, propeptide sequence, consensus translational initiator sequence, signal peptide sequence, and transcription terminator.

17. The method of claim 13, wherein the disrupted gene is disrupted with a selectable marker gene selected from the group consisting of amds (acetamidase), argB (ornithine carbamoyltransferase), bar (phosphinothricin acetyltransferase), hygB (hygromycin phosphotransferase), niaD (nitrate reductase), pyrG (orotidine-5′-phosphate decarboxylase), sC (sulfate adenyltransferase), and trpC (anthranilate synthase); and equivalents thereof.

18. The method of claim 13, wherein the transposon is selected from the group consisting of P elements, LINES, SINES, Ty1, gypsy, Fot1, hAT, Restless, Guest, elements, tn10, Tad-1, Afut-1, and the retrotransposons MAGGY Ty3 and Ty5.

19. The method of claim 13, wherein the DNA sequence is a ribozyme.

20. The method of claim 1, wherein the in vivo recombination occurs by homologous recombination.

21. The method of claim 1, wherein the in vivo recombination occurs by non-homologous recombination.

22. The method of claim 1, wherein the one or more regions of the DNA fragments that are homologous to the DNA sequence are a 5′ region and/or a 3′ region that flank (a) a gene that encodes a polypeptide or an RNA; (b) a gene disrupted with a third nucleic acid sequence; (c) a partially deleted gene; (d) a regulatory control sequence; (e) a recombinantly manipulated version of a gene native or foreign to the filamentous fungal host cell; (f) a transposon; (g) a ribozyme; or (h) a portion of (a), (b), (c), (d), (e), (f) or (g).

23. The method of claim 1, wherein the one or more regions of the DNA fragments that are homologous to the DNA sequence are a 5′ region and/or a 3′ region of (a) a gene that encodes a polypeptide or an RNA; (b) a gene disrupted with a third nucleic acid sequence; (c) a partially deleted gene; (d) a regulatory control sequence; (e) a recombinantly manipulated version of a gene native or foreign to the filamentous fungal host cell; (f) a transposon; (g) a ribozyme; or (h) a portion of (a), (b), (c), (d), (e), (f) or (g).

24. The method of claim 1, wherein the one or more regions of the DNA fragments that are homologous to the DNA sequence are part of a gene native or foreign to the filamentous fungal host cell.

25. The method of claim 13, wherein the hormone or protein with biological activity are selected from the group consisting of insulin, ACTH, glucagon, somatostatin, somatotropin, thymosin, parathyroid hormone, pigmentary hormones, somatomedin, erythropoietin, luteinizing hormone, chorionic gonadotropin, hypothalamic releasing factors, antidiuretic hormones, thyroid stimulating hormone, relaxin, interferon, thrombopoietin, and prolactin.

26. The method of claim 1, wherein at least one of the DNA sequences is a wild-type DNA sequence.

27. The method of claim 1, wherein the regions homologous to the DNA sequence or vector sequence are at least 60% homologous.

28. The method of claim 1, wherein the regions homologous to the DNA sequence or vector sequence are at least 60% homologous.

29. The method of claim 1, wherein the regions homologous to the DNA sequence or vector sequence are at least 70% homologous.

30. The method of claim 1, wherein the regions homologous to the DNA sequence or vector sequence are at least 80% homologous.

31. The method of claim 1, wherein the regions homologous to the DNA sequence or vector sequence are at least 90% homologous.

32. The method of claim 1, wherein the filamentous fungal cell is an Acremonium, Aspergillus, Aureobasidium, Cryptococcus, Filibasidium, Fusarium, Gibberella, Humicola, Magnaporthe, Mucor, Myceliophthora, Myrothecium, Neocallimastix, Neurospora, Paecilomyces, Penicillium, Piromyces, Schizophyllum, Talaromyces, Thermoascus, Thielavia, Tolypocladium, or Trichoderma strain.

33. The method of claim 1, wherein the filamentous fungal cell is an Aspergillus strain.

34. The method of claim 1, wherein the Aspergillus strain is Aspegillus oryzae.

35. The method of claim 1, wherein the Aspergillus strain is Aspergillus niger.

36. The method of claim 1, wherein the replicator sequence is AMAL or ANS1.

37. The method of claim 1, wherein the filamentous fungal host cells further comprise a heterologous gene encoding a recombination protein.

38. The method of claim 35, wherein the gene encoding a recombination protein is selected from the group consisting of: (a) a nucleic acid sequence having at least 70% identity with SEQ ID NO:2, SEQ ID NO:4 or SEQ ID NO:6; (b) a nucleic acid sequence having at least 70% homology with SEQ ID NO:1, SEQ ID NO:3 or SEQ ID NO:5; (c) a nucleic acid sequence which hybridizes under medium stringency conditions with (i) SEQ ID NO:1, SEQ ID NO:3 or SEQ ID NO:5, (ii) the cDNA sequence contained in SEQ ID NO:1, SEQ ID NO:3 or SEQ ID NO:5, or (iii) a complementary strand of (i) or (ii); and (d) a subsequence of (a), (b), or (c), wherein the subsequence encodes a polypeptide fragment which has recombination activity.

39. The method of claim 38, wherein the recombination polypeptide has at least 70% identity with SEQ ID NO: 2, SEQ ID NO:4 or SEQ ID NO:6.

40. The method of claim 39, wherein the recombination polypeptide has at least 75% identity with SEQ ID NO: 2, SEQ ID NO:4 or SEQ ID NO:6.

41. The method of claim 40, wherein the recombination polypeptide has at least 80% identity with SEQ ID NO: 2, SEQ ID NO:4 or SEQ ID NO:6.

42. The method of claim 41, wherein the recombination polypeptide has at least 85% identity with SEQ ID NO: 2, SEQ ID NO:4 or SEQ ID NO:6.

43. The method of claim 42, wherein the recombination polypeptide has at least 90% identity with SEQ ID NO: 2, SEQ ID NO:4 or SEQ ID NO:6.

44. The method of claim 43, wherein the recombination polypeptide has at least 95% identity with SEQ ID NO: 2, SEQ ID NO:4 or SEQ ID NO:6.

45. The method of claim 38, wherein the recombination protein comprises the amino acid sequence of SEQ ID NO:2, SEQ ID NO:4 or SEQ ID NO:6.

46. The method of claim 38, wherein the recombination protein consists of the amino acid sequence of SEQ ID NO:2, SEQ ID NO:4 or SEQ ID NO:6; or a fragment thereof which has recombination activity.

47. The method of claim 46, wherein the recombination protein consists of the amino acid sequence of SEQ ID NO:2, SEQ ID NO:4 or SEQ ID NO:6.

48. The method of claim 47, wherein SEQ ID NO:2 is encoded by SEQ ID NO:1, SEQ ID NO:4 is encoded by SEQ ID NO:3, and SEQ ID NO:6 is encoded by SEQ ID NO:5.

49. The method of claim 38, wherein the nucleic acid sequence of the gene encoding the recombination polypeptide has at least 70% homology with SEQ ID NO:1, SEQ ID NO:3 or SEQ ID NO:5.

50. The method of claim 49, wherein the nucleic acid sequence of the gene encoding the recombination polypeptide has at least 75% homology with SEQ ID NO:1, SEQ ID NO:3 or SEQ ID NO:5.

51. The method of claim 50, wherein the nucleic acid sequence of the gene encoding the recombination polypeptide has at least 80% homology with SEQ ID NO:1, SEQ ID NO:3 or SEQ ID NO:5.

52. The method of claim 51, wherein the nucleic acid sequence of the gene encoding the recombination polypeptide has at least 85% homology with SEQ ID NO:1, SEQ ID NO:3 or SEQ ID NO:5.

53. The method of claim 52, wherein the nucleic acid sequence of the gene encoding the recombination polypeptide has at least 90% homology with SEQ ID NO:1, SEQ ID NO:3 or SEQ ID NO:5.

54. The method of claim 53, wherein the nucleic acid sequence of the gene encoding the recombination polypeptide has at least 95% homology with SEQ ID NO:1, SEQ ID NO:3 or SEQ ID NO:5.

55. The method of claim 38, wherein the first nucleic acid sequence encoding the recombination polypeptide hybridizes under medium stringency conditions with (i) SEQ ID NO:1, SEQ ID NO:3 or SEQ ID NO:5, (ii) the cDNA sequence contained in SEQ ID NO:1, SEQ ID NO:3 or SEQ ID NO:5, or (iii) a complementary strand of (i) or (ii).

56. The method of claim 55, wherein the nucleic acid sequence of the gene encoding the recombination polypeptide hybridizes under medium-high stringency conditions with (i) SEQ ID NO:1, SEQ ID NO:3 or SEQ ID NO:5, (ii) the cDNA sequence contained in SEQ ID NO:1, SEQ ID NO:3 or SEQ ID NO:5, or (iii) a complementary strand of (i) or (ii).

57. The method of claim 56, wherein the nucleic acid sequence of the gene encoding the recombination polypeptide hybridizes under high stringency conditions with (i) SEQ ID NO:1, SEQ ID NO:3 or SEQ ID NO:5, (ii) the cDNA sequence contained in SEQ ID NO:1, SEQ ID NO:3 or SEQ ID NO:5, or (iii) a complementary strand of (i) or (ii).

58. The method of claim 38, wherein the gene is the nucleic acid sequence contained in plasmid pZL1 rdhA13 which is contained in Escherichia coli NRRL B-30503; plasmid pZL1 rdhB6 which is contained in Escherichia coli NRRL B-30504; or plasmid pZL1rdhD17 which is contained in Escherichia coli NRRL B-30505 and plasmid pZL1rdhD10 which is contained in Escherichia coli NRRL B-30506.

59. The method of claim 1, further comprising isolating from the population of recombinant filamentous fungal cells an autonomously replicating plasmid comprising a variant DNA sequence.

60. The method of claim 40, wherein the variant DNA sequence encodes a product with an improved property of interest.

61. The method of claim 41, wherein the improved characteristic is selected from the group consisting of thermostability, thermolability, protease-resistance, pH optimum, pH stability, altered substrate specificity, and increased promoter activity.

62. An autonomously replicating plasmid obtained by the method of claim 1.


SEQUENCE LISTING (58 sequences)

SEQ ID NO: 1 - DNA, 2032 bp, A. oryzae
ttctcggactggatgaagcaagatgcaacgaagcaagctcatgatctgacattagggcccctctcatgtttccttgcattattgttccaatatagcgttgcgtaattgtaggcttacctcttaaggcagcagcctgatgtgcctggaacacgtgacctcgtgacagcctcgacgcgtccagaacctcaacaaattgtttatcgccgtagagtcatacgccattttgccacatcgaccgacttacgaattttaaataagacttctattgtttccaaactggtgactacaaagcagcctttggggtattcgtccgataagaaacaatcctagcgaagaattcatccacagttacaatagaacgcgtcctgtcgcgtcgagtaggctgtgtgcaagaccagtagcagttgcttgattactcttggggctggctagggagacactttacttgcctacctgattgaaaatgacggcggatatggatactcagaatgaatacgatgatagtggacttcccgggcctggagcgcccacgccactttcagctttagaagtgaggaatctctactgtccacacaatacaatattacaagcttgatttagtagtaaaagcccctttcagtgacttgtactgaccattgactcgatagggtgttgcgggattaacgggaagagatatcaaattgtttgtcgatgccggctatcacactgtcgaatcaattgcgtatacgtacgtctttcctcttgaatgtttaaacgaacttgctgcagcaccaaacttggagagcttaaggagatgctgacattggcattggtggtacatcatagaccgaaacgtttactggaacaaatcaaaggtatatcggagcagaaggccaccaaggttttggttgaaggttagtcacctcaactcatggagctcaaccgtctgtaaggattcgtgctgattatacctgataaatagctgccaagcttgtgccaatgggtttcacgactgcaacagaaatgcatgcacgtcgaagtgagctcatatcgatcacgacaggatccaagcaactagatactctcctaggcggtggtatagaaacgggatctattaccgagatattcggagaattcaggacaggtaaaagtcaaatttgccatacgcttgcagtgacttgccagctgccattcgacatgggtggtggggaagggaagtgtctttatattgatactgaagggacatttcgaccggtccgtctgttggcagttgctcaaagatacggacttgttggcgaagaggtactcgataatgtggcctatgcccgcgcttataactcggatcaccagctccagctgctgaaccaggcgtctcaaatgatgtgcgaaactcgtttctcacttcttgtcgtcgactctgctacagcgctatatcggacagattttaacggccgtggtgaactatcgactcgacaaacacatctcgctaaattcatgcgtaccttgcagcgcttggcggatgaatttggtattgccgtcgtcatcaccaaccaggtcgtcgcccaggtcgacggcggtccgagtgcaatgttcaacccagaccccaagaagccaatcggtggaaacattatcgcacacgccagcacgaccaggctgagtctgaaaaaggggagaggagagacccgagtgtgcaagatctatgacagtccctgtctgcccgagagtgactgtctttttgctatcaatgaagatggtattggggatcctagccccaaggacttggaaaacaactgaggagcgatgaagctgtattaattacttacgataccacgatcggtatatgattttacttggtttgttctttagtacatattgtttagtatcttgattttgatagcatacggtgtttgtggtattgtgctagattttatgtgctaattgagataaaagttgatcaataaaaaaagaactatgacttgtatatacaaagaacgtatggtcttctaataatatctatttcgaacgatttgctcttctgtccttccatcaaatt

SEQ ID NO: 2 - protein, 315 aa, A. oryzae
MTADMDTQNEYDDSGLPGPGAPTPLSALEGVAGLTGRDIKLFVDAGYHTVESIAYTPKRLLEQIKGISEQKATKVLVEAAKLVPMGFTTATEMHARRSELISITTGSKQLDTLLGGGIETGSITEIFGEFRTGKSQICHTLAVTCQLPFDMGGGEGKCLYIDTEGTFRPVRLLAVAQRYGLVGEEVLDNVAYARAYNSDHQLQLLNQASQMMCETRFSLLVVDSAEFGIAVVITNQVVAQVDGGPSAMFNPDPKKPIGGNIIAHASTTRLSLKKGRGETRVCKIYDSPCLPESDCLFAINEDGIGDPSPKDLENN

SEQ ID NO: 3 - DNA, 2500 bp, A. oryzae
gtggtatcgcgactgtcggattccgtcacgcgtcgagcagctcagttgacctctgtctatcttcattaccccccactgtgtacgccacgcgcatcggtgtataccaatgataccctttcttaactagtgtaacatttatatatcttttaataatgcccgcgtgagccaccccagtgtgctacgctaccgctgctattaatcccgatctaagctaacgcgtcttagtgttggcgaccaacatcgaggcaatccgacatgcatcatgatgcccaacacgacagacactacatcagcaaacccctttgaggaacgtcctcgccgcatgaatgagtatacagctcgggagatcgccacactgcaagcacggctcgataagaaattaggccccgaatacatctcctctaggccaggcgccgcgggacagagagtccattatctggctgcagacaaatgcattaacctagccaacgaggtctttggtttcaatgggtggtcaagttcgatacaacaaattcagatcgatttcgtatgttctattgataggagcatatctatgttgcgtgtgcccgagatcggacagctgataaatccctcgttgtaacaggttgacgagagcccaaatacggggaagattagcttgggcttgtcagttgtagtgagggtgactctaaaggatgggacctaccatgaggtatacttttgcgtgaatgatatgctccgatgtgcccaaacgctaaccactttggacgacgttaggatatcgggtacggccacattgagaactgtaaagggaaagctgcggctttcgaaaaagcaaagaaagaaggaaccacagatgccctaaagcgtacattgaggaacttcggcaacgtcctgggcaattgcatttatgataaggattacgtatcgaaagtgacgaaagtgaagacagcgcctgtatgtgtttctacggcatattactcgactagactcaggtactaacatgctcccaggcaagatgggacgtggatgaccttcaccgacaccctgatttcgcacccatcaagaaagaaccagttcaacagaagccgatgcaggaggatgatgatctccctcctcgcccgactgatgcgggaaagaacaacagtaactcagccgatactgcctttgatgctgatggagagttcggaagtacgtaacgataaatcaagctaccttgcatgcatttcactaacgatcgcaggtgatttatttgacgaagcggactttggagtcgccgcaactggaaacccagatgaaatagtaatagacccagatacccaaagatttcagcagccaccaacacctctgaaccgtcaaaatggcccagccccgtacaggggccctcaacagcataaccccttagccgctgcaagaccccattccgccattgccacaccatccaaaccagaaagaccgccgaaccaggcagctgccgctagacagataccacctcctgctctgaatggcagaccaaaccctgctgcacccgcccacaacccgcaacacaaccttccaagcggaagaataccaccagctcaaccaagacctaatcaagacacagccatgcccggtgcaagtggtcagatgcccatcaaacgggaacaagttcctaatcccaacgaccccggaacccaggacatgctcccaccaggaagctcaccgatgccatctgcctcattcttctcagctcgagcagtcgatctcctacgtgacaacccacaagcaaacgcagccccggcattcgacccccatgcagaaagcccatccatccgcaagacagctggcgtcgaccacagtaagagcgtcccgatttccaaacccatgcttgccagcgtatcccccgccgccaacaatacccgtgacttcgtcaacccttctcaagatatgcatcggaaaatcggcgctcctagcggaataggcagtcccatgaatcgaggccagacaacctcatcttaccgcccattaacaagaccgaacatcgaccccaagaatgctgtgaatactacagctgcaaaccggggcgtcgggccacaaaatctaaatgggaaacgacctcccctaagtgatgtgactaatgcatccactttaggcggcagcgggcctgctcccattggtggtgcgatagaccctaagaggccgaaaatcaacgacgggcctcttccacaccaacagcaacaacatccgcaataggactcctagcggatttagtatagttacacggcaacataaataatagccatgcttacggggagatcgtccactcattgcatcgttagaggtcatctcatcggtagttcaagacatggcgttcaggattggggtacgggtaaaggagctccgggttaaatgtaatgaatgttcttgatgaatgttatttttgttatattctcttcactccagcttaaagcatccaagacgtgcctccttaggcagttgtgtgactggattgtctacggacactacacagcttgtactatac

SEQ ID NO: 4 - protein, 565 aa, A. oryzae
MMPNTTDTTSANPFEERPRRMNEYTAREIATLQARLDKKLGPEYISSRPGAAGQRVHYLAADKCINLANEVFGFNGWSSSIQQIQIDFVDESPNTGKISLGLSVVVRVTLKDGTYHEDIGYGHIENCKGKAAAFEKAKKEGTTDALKRTLRNFGNVLGNCIYDKDYVSKVTKVKTAPARWDVDDLHRHPDFAPIKKEPVQQKPMQEDDDLPPRPTDAGKNNSNSADTAFDADGEFGSDLFDEADFGVAATGNPDEIVIDPDTQRFQQPPTPLNRQNGPAPYRGPQQHNPLAAARPHSAIATPSKPERPPNQAAAARQIPPPALNGRPNPAAPAHNPQHNLPSGRIPPAQPRPNQDTAMPGASGQMPIKREQVPNPNDPGTQDMLPPGSSPMPSASFFSARAVDLLRDNPQANAAPAFDPHAESPSIRKTAGVDHSKSVPISKPMLASVSPAANNTRDFVNPSQDMHRKIGAPSGIGSPMNRGQTTSSYRPLTRPNIDPKNAVNTTAANRGVGPQNLNGKRPPLSDVTNASTLGGSGPAPIGGAIDPKRPKINDGPLPHQQQQHPQ

SEQ ID NO: 5 - DNA, 2981 bp, A. oryzae
  misc_feature (2932)..(2932): n= a,c,g, or t
gacagcgtgatactttggtgtttagacggccacagggaaacgcgccaagatgtggcaacgcgttgttcatgactctatggaactgacattgactgccaggcatcagcccacctattactgcgtgaaatagaaaggctttctagatagcaccgctacctttaatgtaaggaaaatattaattctgttctctcatgctataaatcgctaacttctcaaggtatcgaccacgaccgagtgtaagggaagatggcggaagcacaccgtcatccaaaaacctccaaaccccgtcctcaaagtccattgaccgcctatcaaaaccattcaaatgccctggatctgctacacccacacggacatcggataaacctgcgaggaaacgaagaaaggtgaattatgcgggggctgatgaaactgtggacgataatagtgaaaagccatacaccaacgaggaacgtttagcactcgccaccagagatgtgaacaggttccctgtgttcaaacctaaagataaagagacaaccttcaaacaacgattcaagatacctttaatcaacaaggcggttgacagctacaatggcgctagggcggcgccaaccttggggatgcgacaaggtgctacatttgtcgtgaaacctctacatgatcctagcggagaatttgcgatagtgctgtatgatccgactgtcgatgatgccgatgagaacagtgaaacgaagttgccggaagatggaaaacccgaagaacaacaacccaagttggacgctccccttgtacacaagagcttagcagacatacttggtcttaagaagaaagttgaaactggtccaagggttccagtcgtgatagacccaaggttggcaaaggttctacgcccacatcaaattgaaggtgtaaaggtaacatgactgttccaatcaatgctctcctgcgatattagactaacgataactgttttctagtttttataccgctgcacaaccggaatggtcgataagaacgcacacggctgtataatggcggatggaatgggactagggaaaacagtatggccgacagacccttcaaaacactaaccccggctgacggcgatagcttcaatgcatctcattgatgtggacattgctcaagcagtctcctgaggcaggcaagacccttatccagaagtgtatcattgcttgtccttcaagtttggttggcaactgggccaatgagctaggtaggtagtgcgccctggatgttttaacacctgctaacaaccacagtgaaatggctaggtaaagatgccatcactccttttgcggtggatggcaaagcttcgaagacagaactcacatctcagatcaagcaatgggctattgcttccggtcgcgccgttgtgagacctgtgctcattgtgtcctacgaaacgctcaggatgtatgttgaagcattgaaggatagccccatagggctacttctttgcgatgaaggtcatcggcttaaaaataaggatagtttaacatggactgcactcaacagtctgaatgtgcaacgtcgtgttatcttgtcaggaacccctattcaaaatgatctttcggaatatttcgccctgctcaacttcgccaacccagatttattagggtcgcagaatgaatttcggaaaaggttcgaattgcctatcctcagaggaagggatgccgcaggatcggacgaagacaaaaagaaaggcgatgaatgtctagctgagctctcaaccatcgtcaacaaattcattatccgccgaacaaatgatatattgacgaaatacttgccagtcaagtatgagcatgttgtcttttgcaatttgtctcaattccaactcgacctttataaccacttcattcagagcccagaaattaggagcttgctcaggggcaaaggaagccagccgcttaaggcaattggccttttgaaaaagctttgcaaccatcctgatctacttaacctttccaccgaccttccaggatgcgaatttgcatttccagaagattacgtgccacctgaggcaagagggcgtgaccgcgatatcaagtcttggtactcggggaaaatgatggttttggatcgaatgctagcacgtatacgccaggacacaaatgacaaaattgttctcattagtaattacacccagacacttgacctgttcgaaaagctatgcagatcgagggggtatggctcgttgagactggacggtactatgaatgtgaataagcggcaaaagctcgtcgacaaattcaacaaccctgacggggaagaatttgtatttctcctcagcagcaaggccggtggatgtggcctcaatctaataggcgccaatcgtctcgtgctgtttgacccagattggaacccagctgctgatcaacaagcattggcacgagtttggcgtgatggtcagaagaaagactgtttcgtgtaccgatttatcgcgaccggctcaattgaggagaagatcttccaacggcagtctcataagcaatcattgtcctcatgcgttgtggattcagcggaagatgttgagcggcatttttctttggagtctctccgcgaactattccaattcaaaccggaaacccgaagtgacacacatgacaccttcaagtgcaagagatgcagaccggatggagcgcaattcatcaaggcgcaggctatgttgtatggcgataccagcacctggaatcactttgttaatgatggcgagaagggtgcccttagcaagatccaggacctgctgatacgacaggagaccggggagagagatgtgtctgcggtattccagtatataagtcactgatctaattcttacaagcgtcgtgttttacactgtctatatgttcaaagcagttgatgctacggcaatcatgagttggcacaattctgggctgtcagtgcagctatttacatttggtagctaggnatatcatgcattcatgcttttgctatcataggctacattaggtctatgg

SEQ ID NO: 6 - protein, 811 aa, A. oryzae
MYRPRPSVREDGGSTPSSKNLQTPSSKSIDRLSKPFKCPGSATPTRTSDKPARKRRKVNYAGADETVDDNSEKPYTNEERLALATRDVNRFPVFKPKDKETTFKQRFKIPLINKAVDSYNGARAAPTLGMRQGATFVVKPLHDPSGEFAIVLYDPTVDDADENSETKLPEDGKPEEQQPKLDAPLVHKSLADILGLKKKVETGPRVPVVIDPRLAKVLRPHQIEGVKFLYRCTTGMVDKNAHGCIMADGMGLGKTLQCISLMWTLLKQSPEAGKTLIQKCIIACPSSLVGNWANELVKWLGKDAITPFAVDGKASKTELTSQIKQWAIASGRAVVRPVLIVSYETLRMYVEALKDSPIGLLLCDEGHRLKNKDSLTWTALNSLNVQRRVILSGTPIQNDLSEYFALLNFANPDLLGSQNEFRKRFELPILRGRDAAGSDEDKKKGDECLAELSTIVNKFIIRRTNDILTKYLPVKYEHVVFCNLSQFQLDLYNHFIQSPEIRSLLRGKGSQPLKAIGLLKKLCNHPDLLNLSTDLPGCEFAFPEDYVPPEARGRDRDIKSWYSGKMMVLDRMLARIRQDTNDKIVLISNYTQTLDLFEKLCRSRGYGSLRLDGTMNVNKRQKLVDKFNNPDGEEFVFLLSSKAGGCGLNLIGANRLVLFDPDWNPAADQQALARVWRDGQKKDCFVYRFIATGSIEEKIFQRQSHKQSLSSCVVDSAEDVERHFSLESLRELFQFKPETRSDTHDTFKCKRCRPDGAQFIKAQAMLYGDTSTWNHFVNDGEKGALSKIQDLLIRQETGERDVSAVFQYISH

SEQ ID NO: 7 - DNA, 20 bp, A. oryzae
  misc_feature (3)..(3): y= c or t
gayaaygtngcntaygcnmg

SEQ ID NO: 8 - DNA, 20 bp, A. oryzae
  misc_feature (3)..(3): n= inosine
ttnggrtcnggrttraacat

SEQ ID NO: 9 - DNA, 20 bp, A. oryzae
  misc_feature (3)..(3): y= c or t
acytgngcnacnacytgrtt

SEQ ID NO: 10 - DNA, 29 bp, A. oryzae
  misc_feature (20)..(20): y= c or t
cgaacgaagtcttcggtttyaayggntgg

SEQ ID NO: 11 - DNA, 30 bp, A. oryzae
  misc_feature (19)..(19): n= a,c,g, or t
cttcatgccgtcggtagtnccytcyttytt

SEQ ID NO: 12 - DNA, 42 bp, A. oryzae
tatcaattcttaattaaggatccaagcttgtttaaacaattc

SEQ ID NO: 13 - DNA, 40 bp, A. oryzae
agttaacaattaattcctaggttcgaacaaatttgttaac

SEQ ID NO: 14 - DNA, 33 bp, A. oryzae
gatacatgttatggagatgttctatcacacaag

SEQ ID NO: 15 - DNA, 29 bp, A. oryzae
caggatcctgcagtattgactactatggt

SEQ ID NO: 16 - DNA, 32 bp, A. oryzae
ctgtttaaactgcagggaggaactgaaaaagg

SEQ ID NO: 17 - DNA, 31 bp, A. oryzae
gttaagcttgcgaaacgcaaataatgtgttg

SEQ ID NO: 18 - DNA, 35 bp, A. oryzae
gttacatgtctccagaacgacgcccggcggacatc

SEQ ID NO: 19 - DNA, 28 bp, A. oryzae
tgaagcttcagatctcggtgacgggcag

SEQ ID NO: 20 - DNA, 33 bp, A. oryzae
ggttaattaaccggcagggaaggccaatgaaag

SEQ ID NO: 21 - DNA, 39 bp, A. oryzae
ccacgcgtatttaaatgtccgggatggatagcactgtgg

SEQ ID NO: 22 - DNA, 37 bp, A. oryzae
ggacgcgtgcggccgcgtaccaggagtacgtcgcagg

SEQ ID NO: 23 - DNA, 28 bp, A. oryzae
ggagatctgcagctgtgtaccaatagac

SEQ ID NO: 24 - DNA, 25 bp, A. oryzae
catttaaatgatgacggcggatatg

SEQ ID NO: 25 - DNA, 27 bp, A. oryzae
gttaattaatcagttgttttccaagtc

SEQ ID NO: 26 - DNA, 27 bp, A. oryzae
agcggccgctcagttgttttccaagtc

SEQ ID NO: 27 - DNA, 28 bp, A. oryzae
atttaaatgatgcccaacacgacagaca

SEQ ID NO: 28 - DNA, 28 bp, A. oryzae
ttaattaactattgcggatgttgttgct

SEQ ID NO: 29 - DNA, 27 bp, A. oryzae
gcggccgcctattgcggatgttgttgc

SEQ ID NO: 30 - DNA, 19 bp, A. oryzae
  misc_feature (3)..(3): y= c or t
gayccngaytggaayccng

SEQ ID NO: 31 - DNA, 20 bp, A. oryzae
  misc_feature (3)..(3): y= c or t
ttyttytgnccrtcnckcca

SEQ ID NO: 32 - DNA, 20 bp, A. oryzae
  misc_feature (3)..(3): y= c or t
aaytayacncaracnytnga

SEQ ID NO: 33 - DNA, 19 bp, A. oryzae
  misc_feature (3)..(3): n= inosine
atnttytcytcdatngtnc

SEQ ID NO: 34 - DNA, 19 bp, A. oryzae
aatgcttgttgatcagcag

SEQ ID NO: 35 - DNA, 33 bp, A. oryzae
gcaagcgcgcgcaatacatggtgttttgatcat

SEQ ID NO: 36 - DNA, 69 bp, A. oryzae
gcctctagatctcccgggcgcgccggcacatgtaccaggtcttaagctcgagctcggtcaccggtggcc

SEQ ID NO: 37 - DNA, 30 bp, A. oryzae
gaatgacttggttgacgcgtcaccagtcac

SEQ ID NO: 38 - DNA, 25 bp, A. oryzae
cttattagtaggttggtacttcgag

SEQ ID NO: 39 - DNA, 37 bp, A. oryzae
gtccccagagtagtgtcactatgtcgaggcagttaag

SEQ ID NO: 40 - DNA, 64 bp, A. oryzae
gtatgtcccttgacaatgcgatgtatcacatgatataattactagcaagggaagccgtgcttgg

SEQ ID NO: 41 - DNA, 59 bp, A. oryzae
cctctagatctcgagctcggtcaccggtggcctccgcggccgctggatccccagttgtg

SEQ ID NO: 42 - DNA, 33 bp, A. oryzae; identical to SEQ ID NO: 35

SEQ ID NO: 43 - DNA, 31 bp, A. oryzae
ttgaattgaaaatagattgatttaaaacttc

SEQ ID NO: 44 - DNA, 25 bp, A. oryzae
ttgcatgcgtaatcatggtcatagc

SEQ ID NO: 45 - DNA, 26 bp, A. oryzae
ttgaattcatgggtaataactgatat

SEQ ID NO: 46 - DNA, 32 bp, A. oryzae
aaatcaatctattttcaattcaattcatcatt

SEQ ID NO: 47 - DNA, 45 bp, A. oryzae
ggatgctgttgactccggaaatttaacggtttggtcttgcatccc

SEQ ID NO: 48 - DNA, 44 bp, A. oryzae
ggtattgtcctgcagacggcaatttaacggcttctgcgaatcgc

SEQ ID NO: 49 - DNA, 26 bp, A. oryzae
tctgtgaggcctatggatctcagaac

SEQ ID NO: 50 - DNA, 27 bp, A. oryzae
gatgctgcatgcacaactgcacctcag

SEQ ID NO: 51 - DNA, 30 bp, A. oryzae
atcggttttatgtcttccaagtcgcaattg

SEQ ID NO: 52 - DNA, 33 bp, A. oryzae
cttggaagacataaaaccgatggaggggtagcg

SEQ ID NO: 53 - DNA, 26 bp, A. oryzae
tctgtgaggcctatggatctcagaac

SEQ ID NO: 54 - DNA, 27 bp, A. oryzae
gatgctgcatgcacaactgcacctcag

SEQ ID NO: 55 - DNA, 33 bp, A. oryzae
cgcggatccaccatgcgtaccgccaagttcgcc

SEQ ID NO: 56 - DNA, 30 bp, A. oryzae
gccccgggttacaggcactgagagtaccag

SEQ ID NO: 57 - DNA, 23 bp, A. oryzae
cgatctcgcagtcccgattcgcc

SEQ ID NO: 58 - DNA, 23 bp, A. oryzae
tccgggagctgcatgtgtcagag